(19) United States
(12) Patent Application Publication
(10) Pub. No.: US 2017/0096649 A1
(43) Pub. Date: Apr. 6, 2017

(54) TRANSGENIC NUCLEASE SYSTEMS AND METHODS

(71) Applicant: Agenovir Corporation, South San Francisco, CA (US)

(72) Inventor: Stephen R. Quake, Stanford, CA (US)

(21) Appl. No.: 15/278,841

(22) Filed: Sep. 28, 2016

Related U.S. Application Data
(60) Provisional application No. 62/236,271, filed on Oct. 2, 2015.

(51) Int. Cl. C12N 9/22; A01K 67/027; C12N 15/82

(52) U.S. Cl. CPC C12N 9/22; C12Y 301/00; C12N 15/8222; A01K 67/0275; A01K 2217/052; A01K 2217/206

(57) ABSTRACT

The invention provides transgenic organisms that include a transgene that codes for a product that can be used to digest foreign nucleic acid. The transgene can code for a targeting nuclease, a guide sequence, or other components of a guided nuclease system. Expression of the transgene causes the organism to express an active targeting nuclease that targets and digests foreign nucleic acid. The targeting nuclease targets the foreign nucleic acid specifically and avoids targeting the organism's native genetic material.

### RELATED APPLICATION

This application claims the benefit of and priority to U.S. Provisional No. 62/236,271, filed Oct. 2, 2015, which is incorporated by reference in its entirety.

### TECHNICAL FIELD

The invention relates to transgenic agricultural organisms.

### BACKGROUND

Infections are a significant problem in agriculture. Virus epidemics of plants and livestock have increased steadily since the Neolithic period. As humans became dependent on agriculture and farming, diseases such as potyvirus of potatoes and rinderpest of cattle had devastating consequences. One of the first viruses to be discovered, tobacco mosaic virus, has cost billions of dollars in crop losses. Another important pathogen of animals and plants are the viruses of the family Rhabdoviridae, which includes rabies. Another example is a virus from family Potyviridae known as the Tulip breaking virus (TBV). TBV has dramatic effects on the colors of tulip petals, which in the Netherlands in the 17th century contributed to the skyrocketing prices of rare tulip bulbs, but which also retards the ability of the tulip to propagate.

Viruses of livestock are myriad. For example, different species of the Alphaviruses variously infect horses and farmed fish. The Pestiviruses are associated with swine fever and bovine viral diarrhea/Mucosal disease (BVD/MD). The Arteriviridae family includes equine arteritis virus (EAV) and porcine reproductive and respiratory syndrome virus (PRRSV). Other viruses or families of viruses with dramatic effects on agriculture include coronaviruses, paramyxoviruses, Hendra and Nipah virus, avian influenza, Bluetongue virus (BTV), Porcine Circoviruses (PCV), and African swine fever. There is quite a wide variety of viruses that have a devastating financial effect on agricultural and that do significant harm to animal welfare.

### SUMMARY

The invention provides transgenic organisms that include a transgene that codes for a product that can be used to digest foreign nucleic acid. The transgene can code for a targeting nuclease, a guide sequence, or other components of a guided nuclease system. The living organism expresses the transgene itself, allowing it to express an active targeting nuclease that targets and digests foreign nucleic acid. The nuclease is preferably a programmable nuclease. The nuclease can be, for example, a zinc finger nuclease, a meganuclease, a TALENs, Cpf1, PfAgo, or NgAgo, and is preferably Cas9. The targeting nuclease targets the foreign nucleic acid specifically and avoids targeting the organism's native genetic material. For example, the transgene may encode a Cas9 enzyme that, when expressed, uses a guide RNA sequence complementary to the foreign nucleic acid to bind to, and make cuts in, that foreign nucleic acid. The guide sequence may be encoded by the transgene in the organism in the first instance or may be integrated into a CRISPR/Cas9 complex that includes the transgene within the organism's somatic cells in response to infection by the operation of the CRISPR/Cas9 machinery. Where the guide sequence is complementary to a target within viral nucleic acid and has no corresponding complementary portion within the organism's genome, the targeting nuclease may digest viral genetic material, thereby protecting the organism from the adverse effects of a viral infection.

Transgene expression may be constitutive or may be controlled by a suitable control mechanism, such as a tissue-specific promoter or controlled by an exogenous agent such as a small molecule. While discussed above in terms of digesting viral nucleic acid, the transgene may be activated or expressed to digest or cut any foreign nucleic acid including, for example, from bacteria or other parasites. Additionally or alternatively, the transgene product may in fact target features of the organism's genome, e.g., to initiate expression or repression of some gene product at some point in the organism's life. Since the transgene can specifically digest foreign nucleic acid such as viral genetic material without interfering with the organism's genome, the organism can have innate protection from the adverse effects of an infection. Since the transgenic organism carries innate protection against infection, the use of the organism in agriculture can avoid the devastating financial impacts and the harms to animal welfare that have characterized agriculture for millennia.

In certain aspects, the invention provides a method of making a non-human transgenic organism. The method includes introducing into a cell a transgene encoding a targeting nuclease, integrating the transgene into heritable genetic material of the cell, and growing the cell into an organism for agricultural use, wherein cells of the organism include the transgene. In some embodiments, the organism is a mammal for livestock and the cell comprises an oocyte or a cell of an embryo and wherein growing the cell into the organism includes transfer of the oocyte or embryo into a recipient female. The targeting nuclease may include Cas9 endonuclease under control of a promoter. In certain embodiments, the organism is a plant and the targeting nuclease comprises Cas9 endonuclease. Thus preferably the organism is a plant crop or mammalian livestock and the targeting nuclease includes Cas9 endonuclease.

The transgene may also encode at least one guide sequence that, when transcribed into a guide RNA, guides the Cas9 endonuclease to digest nucleic acid foreign to the organism. Preferably, the guide RNA has no match according to a predetermined similarity criteria within the genome. For example, the similarity criteria may require that the guide RNA has no match >60% within the genome.

Aspects of the invention provide a non-human transgenic organism comprising a transgene. The transgene includes nucleic acid that encodes a targeting nuclease that can be activated to digest foreign nucleic acid. The organism may include a feature that promotes expression of the transgene. For example, the feature that promotes expression may be a promoter-enhancer cassette that selectively favors expression of the targeting nuclease within a certain tissue or cell type of the organism. In some embodiments, the transgene is under the control of an inducible promoter. Suitable inducible promoters include, for example, the tetracycline on system or the tetracycline off system. In certain embodiments, the nuclease is expressed constitutively. In some embodiments, the nuclease is one selected from the group consisting of a zinc-finger nuclease, a transcription activator-like effector nuclease, and a meganuclease. In preferred embodiments, the nuclease comprises Cas9 endonuclease and the targeting sequence comprises a guide RNA. The organism may be a plant crop or mammalian livestock, and the targeting nuclease may use a targeting sequence to target and digest the foreign nucleic acid without digesting a genome of the organism.

In some embodiments, the guide RNA has no match according to a predetermined similarity criteria within the genome of the transgenic organism (e.g., the guide RNA has no match >60% within the genome). In certain embodiments, the targeting sequence is encoded adjacent the targeting nuclease within a complex in the transgene, and the complex is transcribed together as a single primary transcript. Activation of the targeting nuclease includes causing the complex to be transcribed. The nuclease may be activated by administration of an agent such as a small molecule. In some embodiments, activating the targeting nuclease includes causing expression of the targeting nuclease from the transgene and causing the targeting nuclease to digest viral foreign nucleic acid.

In some embodiments, the organism is a plant such as corn, wheat, maize, rapeseed, soybean, sunflower, barley, sorghum, potato, or rice. In certain embodiments, the organism is an animal (e.g., cattle, horse, goat, sheep, swine, and poultry).

Aspects of the invention provide a seed for a crop plant. The seed includes at least one transgene in its heritable genetic material, in which the transgene encodes a targeting nuclease. The targeting nuclease may be a Cas9 endonuclease. The crop plant may be, for example, corn, wheat, maize, rapeseed, soybean, sunflower, barley, sorghum, potato, and rice. In some embodiments, the transgene also encodes at least one guide sequence that, when transcribed into a guide RNA, guides the Cas9 endonuclease to digest nucleic acid foreign to the crop plant.

### BRIEF DESCRIPTION OF THE DRAWINGS

FIG. 1 diagrams a method for making a non-human transgenic organism.

FIG. 2 shows a composition for introducing a transgene into a cell.

FIG. 3 diagrams a vector according to certain embodiments.

FIG. 4 gives results of digesting foreign nucleic acid.

FIG. 5 shows use of zinc-finger targeting nuclease.

FIG. 6 describes an exemplary method for selecting a gRNA.

FIG. 7 outlines a similarity criteria according to certain embodiments.

FIG. 8 diagrams the avian flu virus genome for targets for cleavage.

FIG. 9 shows a genome of a Bluetongue virus (BTV) for targeting.

FIG. 10 diagrams the tobacco mosaic virus genetic material.

FIG. 11 shows parts of the genome of banana bunchy top virus (BBTV).

FIG. 12 shows a gel resulting from a CRISPR assay.

FIG. 13 shows a composition that includes an EGFP marker fused after the Cas9 protein.

FIG. 14 shows gRNA targets along a reference genome.

FIG. 15 gives genome context around guide RNA sgEBV3/4/5 and PCR primer locations.

FIG. 16 shows large deletions induced by sgEBV3/5 and sgEBV4/5.

FIG. 17 shows that Sanger sequencing confirmed genome cleavage and repair ligation.

### DETAILED DESCRIPTION

Aspects of the invention relate to agricultural/biological (agbio) applications of targetable nucleases and particularly to transgenic organisms such as plants or animals. In some embodiments, the invention provides an organism such as an animal or plant, or a seed for a plant, that itself expresses a targeting nuclease. Additionally, the invention provides methods of creating a transgenic organism that expresses a targeting nuclease. In some embodiments, the organism uses the targeting nuclease to cleave foreign nucleic acid. Additionally or alternatively, a transgenic organism of the invention can use a targeting nuclease to affect gene expression (e.g., by interfering with a promotor or effectively performing a knock-out or knock-in via the transgene). The transgenic organism can express a targeting nuclease, such as Cas9, either in every cell or in tissue specific ways. It can also be expressed constitutively or conditionally, e.g., externally inducible by small molecule activation.

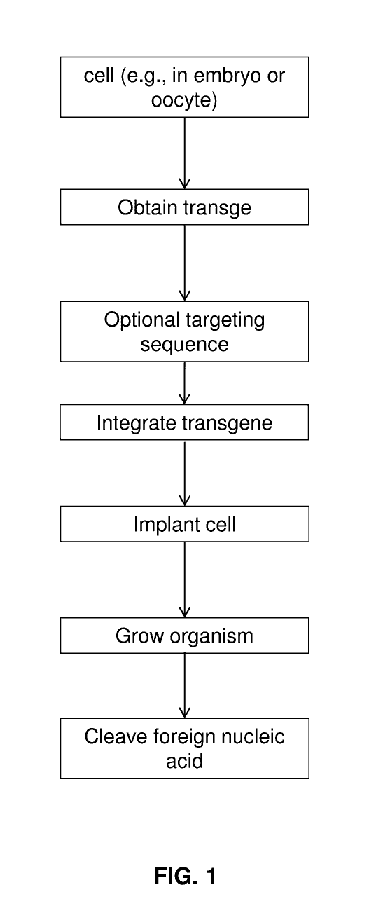

FIG. 1 diagrams a method of making a non-human transgenic organism. The ability to introduce genes and/or other DNA sequences into the germline or somatic cells of organisms such as mammalian livestock or plants results in germline changes that are inherited by the offspring of the animals and all cells of these offspring inherit the introduced transgene. In the depicted method, a cell such as an oocyte or a cell within an embryo is addressed. A transgene is obtained to be integrated into the cell. The transgene may include a gene for a targeting nuclease and may optionally further include a targeting sequence. The transgene is introduced into the cell and the organism is grown to create the transgenic organism. In the preferred embodiment, the transgenic organism may express the transgene to cleave foreign nucleic acid.

### Transgenic Organism

The production of transgenic animals is commonly done in various ways. Transgenic organisms may be produced by targeted insertion of DNA by homologous recombination in embryonic stem (ES) cells or by pronuclear injection of a fertilized ovum and integration of DNA. It is also possible to introduce a transgene via vector such as a plasmid or virus. For example, retroviral vector systems are based on lentiviruses, a small subgroup of the retroviruses.

Several methods for introducing foreign DNA into the germline of mammals have been developed. The techniques allow the mixing of cells from different embryos, i.e. chimera production, introducing pluripotent cells such as ES cells into developing embryos, micro-injecting DNA, and infection by retroviruses. Some techniques include removing fertilized eggs or early embryos, culturing them in vitro and then returning them to recipient mothers where further embryogenesis can proceed. In other embodiments, a lentiviral vector can be introduced throughout the development of the organism. Thus in one embodiment the cell is a perinatal cell, which could be an embryonic cell (e.g., in utero). Preferably the cell is an oocyte, an oviduct cell, an ovarian cell, an ovum, an ES cell, a blastocyte, a spermatocyte, a spermatid, a spermatozoa, or a spermatogonia.

It is possible to insert a foreign gene into mammalian livestock embryos or plant germline cells, and for these genes to be incorporated into the genome of the resulting animal. Insertion of the foreign genes has been carried out mechanically (by microinjection), and with the aid of retrovirus vectors. See e.g., Huszar et al., 1985, Insertion of a bacterial gene into the mouse germ line using an infectious retrovirus vector PNAS 82:8587-8591, incorporated by reference.

The introduced transgene can be sexually transmitted through subsequent generations and are frequently expressed in the animal. In some instances the proteins encoded by the foreign genes are expressed in specific tissues. For example, the metallothionein promoter has been used to direct the expression of the rat growth hormone gene in the liver tissue of transgenic mice (Palmiter et al., 1982, Dramatic growth of mice that develop from eggs microinjected with metallothionein-growth hormone fusion genes, Nature 300:611-615). Another example is the elastase promoter, which has been shown to direct the expression of foreign genes in the pancreas (Ornitz et al., 1985 Nature 313:600). Developmental control of gene expression has also been achieved in transgenic animals, i.e., the foreign gene is transcribed only during a certain time period, and only in a particular tissue. For example, Magram et al. (1985, Nature 315:338) demonstrated developmental control of genes under the direction of a globin promoter; and Krumlauf et al. (1985, Mol. Cell. Biol. 5:1639) demonstrated similar results using the alpha-feto protein mini-gene.

The described methods can be used to generate transgenic, non-human plants or animals or site specific gene modifications in cell lines. Transgenic cells include one or more nucleic acids according to the subject invention present as a transgene, where included within this definition are the parent cells transformed to include the transgene and the progeny thereof.

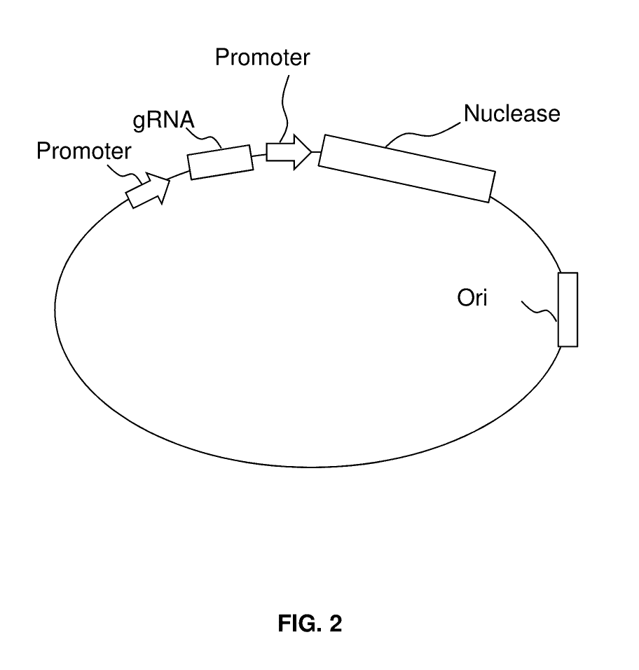

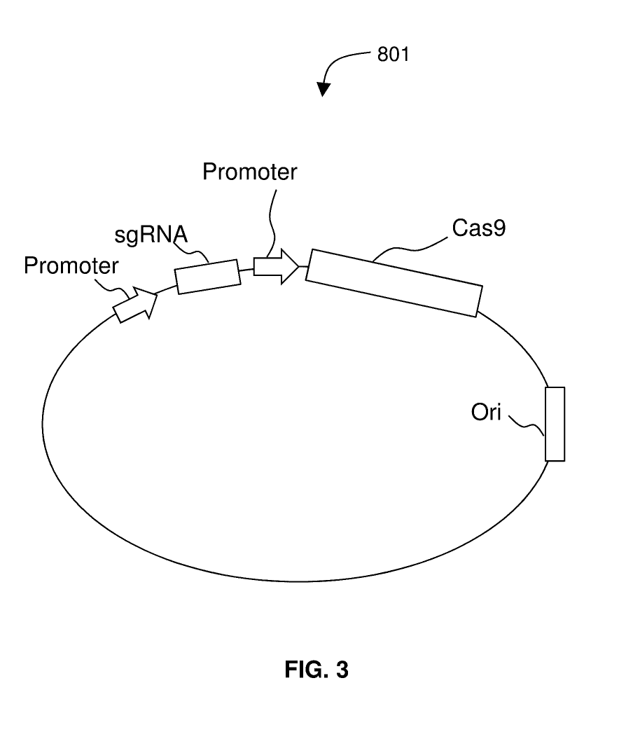

A transgenic animal may be made starting with stem cells. An ES cell line may be employed, or embryonic cells may be obtained freshly from a host, e.g. cow, pig, chicken, etc. Such cells are grown on an appropriate fibroblast-feeder layer or grown in the presence of leukemia inhibiting factor (LIF). When ES or embryonic cells are transformed (e.g., using a vector of FIG. 2 or FIG. 3), they may be used to produce transgenic animals. After transformation, the cells are plated onto a feeder layer in an appropriate medium. Cells containing the construct may be detected by employing a selective medium. After sufficient time for colonies to grow, they are picked and analyzed for the occurrence of homologous recombination or integration of the construct. Those colonies that are positive may then be used for embryo manipulation and blastocyst injection. Blastocysts are obtained from 4 to 6 week old super-ovulated females. The ES cells are trypsinized, and the modified cells are injected into the blastocoel of the blastocyst. After injection, the blastocysts are returned to each uterine horn of pseudo-pregnant females. Females are then allowed to go to term and the resulting offspring screened for the construct. By providing for a different phenotype of the blastocyst and the genetically modified cells, chimeric progeny can be readily detected. The chimeric animals are screened for the presence of the modified gene and males and females having the modification may be mated to produce homozygous progeny. The transgenic animals may be any non-human livestock mammal or any other suitable animal.

Transgenic plants may be produced in a similar manner. Methods of preparing transgenic plant cells and plants are described in U.S. Pat. Nos. 5,767,367; 5,750,870; 5,739,409; 5,689,049; 5,689,045; 5,674,731; 5,656,466; 5,633,155; 5,629,470; 5,595,896; 5,576,198; 5,538,879; 5,484,956; the disclosures of which are herein incorporated by reference. Methods of producing transgenic plants are also reviewed in Plant Biochemistry and Molecular Biology (Eds. Lea & Leegood, John Wiley & Sons)(1993) pp 275-295. In brief, a suitable plant cell or tissue is harvested, depending on the nature of the plant species. As such, in certain instances, protoplasts will be isolated, where such protoplasts may be isolated from a variety of different plant tissues, e.g. leaf, hypoctyl, root, etc. For protoplast isolation, the harvested cells are incubated in the presence of cellulases in order to remove the cell wall, where the exact incubation conditions vary depending on the type of plant and/or tissue from which the cell is derived. The resultant protoplasts are then separated from the resultant cellular debris by sieving and centrifugation. Instead of using protoplasts, embryogenic explants comprising somatic cells may be used for preparation of the transgenic host. Following cell or tissue harvesting, exogenous DNA of interest is introduced into the plant cells, where a variety of different techniques are available for such introduction. With isolated protoplasts, the opportunity arise for introduction via DNA-mediated gene transfer protocols, including: incubation of the protoplasts with naked DNA, e.g. plasmids, comprising the exogenous coding sequence of interest in the presence of polyvalent cations, e.g. PEG or PLO; and electroporation of the protoplasts in the presence of naked DNA comprising the exogenous sequence of interest. Protoplasts that have successfully taken up the exogenous DNA are then selected, grown into a callus, and ultimately into a transgenic plant through contact with the appropriate amounts and ratios of stimulatory factors, e.g. auxins and cytokinins. With embryogenic explants, a convenient method of introducing the exogenous DNA in the target somatic cells is through the use of particle acceleration or “gene-gun” protocols. The resultant explants are then allowed to grow into chimera plants, cross-bred and transgenic progeny are obtained. Instead of the naked DNA approaches described above, another convenient method of producing transgenic plants is Agrobacterium mediated transformation. With Agrobacterium mediated transformation, co-integrative or binary vectors comprising the exogenous DNA are prepared and then introduced into an appropriate Agrobacterium strain, e.g. A. tumefaciens. The resultant bacteria are then incubated with prepared protoplasts or tissue explants, e.g. leaf disks, and a callus is produced. The callus is then grown under selective conditions, selected and subjected to growth media to induce root and shoot growth to ultimately produce a transgenic plant. Although in general the techniques mentioned herein are well known in the art, reference may be made in particular to Sambrook et al., Molecular Cloning, A Laboratory Manual (1989) and Ausubel et al., Short Protocols in Molecular Biology (1999) 4th Ed, John Wiley & Sons, Inc.

### Vectors for Introducing Transgene Into Cell

Methods of the invention may include using a vector to introduce a transgene into a cell such as an oocyte or a cell within an embryo.

FIG. 2 shows a composition for introducing a transgene into a cell. The composition preferably includes a DNA strand (circular or linear, here shown as circularized) that includes at least nuclease gene and at least one targeting sequence (labelled gRNA in FIG. 2). The composition may include an origin of replication such as an HPV origin. Preferably, the composition includes one or more promoters, any or all of which may be specific to keratinocytes. Any suitable promoter or enhancer may be used that results in expression within keratinocytes. For example, a nuclease may be provided within a vector (e.g., a plasmid) that includes one or more inducible promoters such as metallothionein (MT) and 1,24-vitamin D(3)(OH)(2) dehydroxylase (VDH) promoters responded to the inducing agents, Cadmium and 1,25-vitamin D(3)(OH)(2) (VitD(3)), respectively. In one embodiment, the nuclease is Cas9.

FIG. 3 diagrams a vector according to certain embodiments. The vector shown in FIG. 3 may be transfected into an oocyte or a germ cell that is then matured into an agricultural organism. When the organism grows, it will express active Cas9, which may then digest foreign nucleic acid.

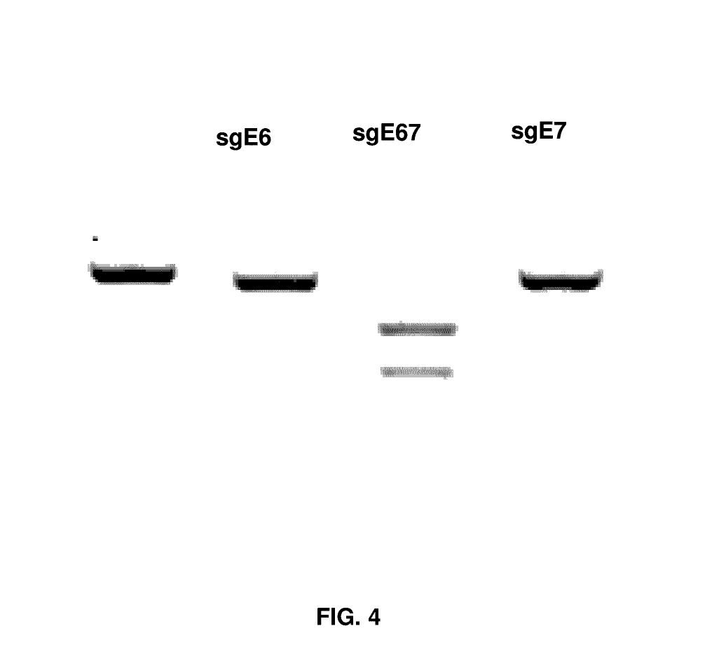

FIG. 4 gives results of digesting foreign nucleic acid. The nuclease forms a complex with the gRNA (e.g., crRNA+tracrRNA or sgRNA). The complex cuts the viral nucleic acid in a targeted fashion to incapacitate the viral genome. The Cas9 endonuclease causes a double strand break in the viral genome. By targeted several locations along the viral genome and causing not a single strand break, but a double strand break, the genome is effectively cut a several locations along the genome. In a preferred embodiment, the double strand breaks are designed so that small deletions are caused, or small fragments are removed from the genome so that even if natural repair mechanisms join the genome together, the genome is render incapacitated.

A transgenic organism (e.g., crop plant or livestock mammal) may be produced using a non-primate lentiviral expression vector. Some vectors used in recombinant DNA techniques allow entities, such as a segment of DNA (such as a heterologous DNA segment, such as a heterologous cDNA segment), to be transferred into a host cell for the purpose of replicating the vectors comprising a segment of DNA. Examples of vectors used in recombinant DNA techniques include but are not limited to plasmids, chromosomes, artificial chromosomes or viruses. In a typical vector for use in the method of the present invention, at least part of one or more protein coding regions essential for replication may be removed from the virus. This makes the viral vector replication-defective. Portions of the viral genome may also be replaced by a library encoding e.g., a targeting nuclease operably linked to a regulatory control region and a reporter moiety in the vector genome in order to generate a vector comprising candidate transgenes which is capable of transducing a target cell and/or integrating its genome into the genome. Lentivirus vectors are part of a larger group of retroviral vectors. A detailed list of lentiviruses may be found in Coffin et al (“Retroviruses” 1997 Cold Spring Harbor Laboratory Press Eds: J M Coffin, S M Hughes, H E Varmus pp 758-763). In brief, lentiviruses can be divided into primate and non-primate groups. Examples of primate lentiviruses include but are not limited to: the human immunodeficiency virus (HIV) and the simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV) and the more recently described feline immunodeficiency virus (FIV) and bovine immunodeficiency virus (BIV). A distinction between the lentivirus family and other types of retroviruses is that lentiviruses have the capability to infect both dividing and non-dividing cells (Lewis et al 1992 EMBO. J 11: 3053-3058; Lewis and Emerman 1994 J. Virol. 68: 510-516). In contrast, other retroviruses—such as MLV—are unable to infect non-dividing or slowly dividing cells such as those that make up, for example, muscle, brain, lung and liver tissue.

A “non-primate” vector, as used herein in some aspects of the present invention, refers to a vector derived from a virus which does not primarily infect primates, especially humans. Thus, non-primate virus vectors include vectors which infect non-primate mammals, such as dogs, sheep and horses, reptiles, birds and insects. The non-primate lentivirus may be any member of the family of lentiviridae which does not naturally infect a primate and may include a feline immunodeficiency virus (FIV), a bovine immunodeficiency virus (BIV), a caprine arthritis encephalitis virus (CAEV), a Maedi visna virus (MVV) or an equine infectious anaemia virus (EIAV). Preferably the lentivirus is an EIAV. Equine infectious anaemia virus infects all equidae resulting in plasma viremia and thrombocytopenia (Clabough, et al. 1991. J Virol. 65:6242-51). Virus replication is thought to be controlled by maturation of monocytes into macrophages.

In one embodiment the viral vector is derived from EIAV. EIAV has the simplest genomic structure of the lentiviruses and is particularly preferred for use in the present invention. In addition to the gag, pol and env genes EIAV encodes three other genes: tat, rev, and S2. Tat acts as a transcriptional activator of the viral LTR (Derse and Newbold 1993 Virology. 194:530-6; Maury, et al 1994 Virology. 200:632-42) and Rev regulates and coordinates the expression of viral genes through rev-response elements (RRE) (Martarano et al 1994 J Virol. 68:3102-11). The mechanisms of action of these two proteins are thought to be broadly similar to the analogous mechanisms in the primate viruses (Martano et al ibid). The function of S2 is unknown. In addition, an EIAV protein, Ttm, has been identified that is encoded by the first exon of that spliced to the env coding sequence at the start of the transmembrane protein.

The viral RNA of this aspect of the invention is transcribed from a promoter, which may be of viral or non-viral origin, but which is capable of directing expression in a eukaryotic cell such as a mammalian cell. Optionally an enhancer is added, either upstream of the promoter or downstream. The RNA transcript is terminated at a polyadenylation site which may be the one provided in the lentiviral 3′ LTR or a different polyadenylation signal.

A DNA transcription unit comprising a promoter and optionally an enhancer capable of directing expression of a non-primate lentiviral vector genome may be used. Transcription units as described herein comprise regions of nucleic acid containing sequences capable of being transcribed. The sequences may be in the sense or antisense orientation with respect to the promoter. Antisense constructs can be used to inhibit the expression of a gene in a cell according to well-known techniques. Nucleic acids may be, for example, ribonucleic acid (RNA) or deoxyribonucleic acid (DNA) or analogues thereof. Sequences encoding mRNA will optionally include some or all of 5′ and/or 3′ transcribed but untranslated flanking sequences naturally, or otherwise, associated with the translated coding sequence. It may optionally further include the associated transcriptional control sequences normally associated with the transcribed sequences, for example transcriptional stop signals, polyadenylation sites and downstream enhancer elements. Nucleic acids may comprise cDNA or genomic DNA (which may contain introns).

The basic structure of a retrovirus genome is a 5′ LTR and a 3′ LTR, between or within which are located a packaging signal to enable the genome to be packaged, a primer binding site, integration sites to enable integration into a host cell genome and gag, pol and env genes encoding the packaging components—these are polypeptides required for the assembly of viral particles. More complex retroviruses have additional features, such as rev and RRE sequences in HIV, which enable the efficient export of RNA transcripts of the integrated provirus from the nucleus to the cytoplasm of an infected target cell.

In the provirus, these genes are flanked at both ends by regions called long terminal repeats (LTRs). The LTRs are responsible for proviral integration, and transcription. LTRs also serve as enhancer-promoter sequences and can control the expression of the viral genes. Encapsidation of the retroviral RNAs occurs by virtue of a psi sequence.

The LTRs themselves are identical sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses. In a defective retroviral vector genome gag, pol and env may be absent or not functional. The R regions at both ends of the RNA are repeated sequences. U5 and U3 represent unique sequences at the 5′ and 3′ ends of the RNA genome respectively.

The retroviral vector employed in the aspects of the present invention may be derived from or may be derivable from any suitable retrovirus. A large number of different retroviruses have been identified. Examples include: murine leukemia virus (MLV), human immunodeficiency virus (HIV), human T-cell leukemia virus (HTLV), mouse mammary tumour virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV). A detailed list of retroviruses may be found in Coffin et al., 1997, “retroviruses”, Cold Spring Harbour Laboratory Press Eds: J M Coffin, S M Hughes, H E Varmus pp 758-763.

### Targetable Nuclease

Methods of the invention include creating a transgenic organism that expresses a targeting nuclease. Any suitable targeting nuclease can be used including, for example, zinc-finger nucleases (ZFNs), transcription activator-like effector nucleases (TALENs), clustered regularly interspaced short palindromic repeat (CRISPR) nucleases, meganucleases, other endo- or exo-nucleases, or combinations thereof. See Schiffer, 2012, Targeted DNA mutagenesis for the cure of chronic viral infections, J Virol 88(17):8920-8936, incorporated by reference. In certain embodiments, the targeting nuclease may be a DNA-guided nuclease (e.g., a Pyrococcus furiosus Argonaute (PfAgo) or Natronobacterium gregoryi Argonaute (NgAgo). The targeting nuclease may be a high-fidelity Cas9 (hi-fi Cas9), e.g., as described in Kleinstiver et al., 2016, High-fidelity CRISPR-Cas9 nucleases with no detectable genome-wide off-target effects, Nature 529:490-495, incorporated by reference.

CRISPR methodologies employ a nuclease, CRISPR-associated (Cas9), that complexes with small RNAs as guides (gRNAs) to cleave DNA in a sequence-specific manner upstream of the protospacer adjacent motif (PAM) in any genomic location. CRISPR may use separate guide RNAs known as the crRNA and tracrRNA. These two separate RNAs have been combined into a single RNA to enable site-specific mammalian genome cutting through the design of a short guide RNA. Cas9 and guide RNA (gRNA) may be synthesized by known methods. Cas9/guide-RNA (gRNA) uses a non-specific DNA cleavage protein Cas9, and an RNA oligo to hybridize to target and recruit the Cas9/gRNA complex. See Chang et al., 2013, Genome editing with RNA-guided Cas9 nuclease in zebrafish embryos, Cell Res 23:465-472; Hwang et al., 2013, Efficient genome editing in zebrafish using a CRISPR-Cas system, Nat. Biotechnol 31:227-229; Xiao et al., 2013, Chromosomal deletions and inversions mediated by TALENS and CRISPR/Cas in zebrafish, Nucl Acids Res 1-11, each incorporated by reference.

CRISPR (Clustered Regularly Interspaced Short Palindromic Repeats) is found in bacteria and is believed to protect the bacteria from phage infection. It has recently been used as a means to alter gene expression in eukaryotic DNA, but has not been proposed as an anti-viral therapy or more broadly as a way to disrupt genomic material. Rather, it has been used to introduce insertions or deletions as a way of increasing or decreasing transcription in the DNA of a targeted cell or population of cells. See for example, Horvath et al., Science (2010) 327:167-170; Terns et al., Current Opinion in Microbiology (2011) 14:321-327; Bhaya et al. Annu Rev Genet (2011) 45:273-297; Wiedenheft et al. Nature (2012) 482:331-338); Jinek Met al. Science (2012) 337:816-821; Cong L et al. Science (2013) 339:819-823; Jinek M et al. (2013) eLife 2:e00471; Mali P et al. (2013) Science 339:823-826; Qi L S et al. (2013) Cell 152:1173-1183; Gilbert L A et al. (2013) Cell 154:442-451; Yang H et al. (2013) Cell 154:1370-1379; and Wang H et al. (2013) Cell 153:910-918), each incorporated by reference.

In an aspect of the invention, the Cas9 endonuclease causes a break one or more locations in foreign nucleic acid. These two double strand breaks may cause a fragment of the genome to be deleted. Even if repair pathways anneal the two ends, there will still be a deletion in the genome. One or more deletions using the mechanism will incapacitate the viral genome. The result is that the transgenic organism will be free of viral infection.

In embodiments of the invention, nucleases cleave the genome of a target virus. A nuclease is an enzyme capable of cleaving the phosphodiester bonds between the nucleotide subunits of nucleic acids. Endonucleases are enzymes that cleave the phosphodiester bond within a polynucleotide chain. Some, such as Deoxyribonuclease I, cut DNA relatively nonspecifically (without regard to sequence), while many, typically called restriction endonucleases or restriction enzymes, cleave only at very specific nucleotide sequences. In a preferred embodiment of the invention, the Cas9 nuclease is incorporated into the compositions and methods of the invention, however, it should be appreciated that any nuclease may be utilized.

In preferred embodiments of the invention, the Cas9 nuclease is used to cleave the genome. The Cas9 nuclease is capable of creating a double strand break in the genome. The Cas9 nuclease has two functional domains: RuvC and HNH, each cutting a different strand. When both of these domains are active, the Cas9 causes double strand breaks in the genome.

In some embodiments of the invention, insertions into the genome can be designed to cause incapacitation, or altered genomic expression. Additionally, insertions/deletions are also used to introduce a premature stop codon either by creating one at the double strand break or by shifting the reading frame to create one downstream of the double strand break. Any of these outcomes of the NHEJ repair pathway can be leveraged to disrupt the target gene. The changes introduced by the use of the CRISPR/gRNA/Cas9 system are permanent to the genome.

In some embodiments of the invention, at least one cut or insertion is caused by the CRISPR/gRNA/Cas9 complex. In a preferred embodiment, numerous insertions are caused in the genome, thereby incapacitating the virus. In an aspect of the invention, the number of insertions lowers the probability that the genome may be repaired.

In some embodiments of the invention, at least one deletion is caused by the CRISPR/gRNA/Cas9 complex. In a preferred embodiment, numerous deletions are caused in the genome, thereby incapacitating the virus. In an aspect of the invention, the number of deletions lowers the probability that the genome may be repaired. In a highly-preferred embodiment, the CRISPR/Cas9/gRNA system of the invention causes significant genomic disruption, resulting in effective destruction of the viral genome, while leaving the host genome intact.

TALENs uses a nonspecific DNA-cleaving nuclease fused to a DNA-binding domain that can be to target essentially any sequence. For TALEN technology, target sites are identified and expression vectors are made. Linearized expression vectors (e.g., by Notl) may be used as template for mRNA synthesis. A commercially available kit may be use such as the mMESSAGE mMACHINE SP6 transcription kit from Life Technologies (Carlsbad, Calif.). See Joung & Sander, 2013, TALENs: a widely applicable technology for targeted genome editing, Nat Rev Mol Cell Bio 14:49-55, incorporated by reference.

TALENs and CRISPR methods provide one-to-one relationship to the target sites, i.e. one unit of the tandem repeat in the TALE domain recognizes one nucleotide in the target site, and the crRNA, gRNA, or sgRNA of CRISPR/Cas system hybridizes to the complementary sequence in the DNA target. Methods can include using a pair of TALENs or a Cas9 protein with one gRNA to generate double-strand breaks in the target. The breaks are then repaired via non-homologous end-joining or homologous recombination (HR).

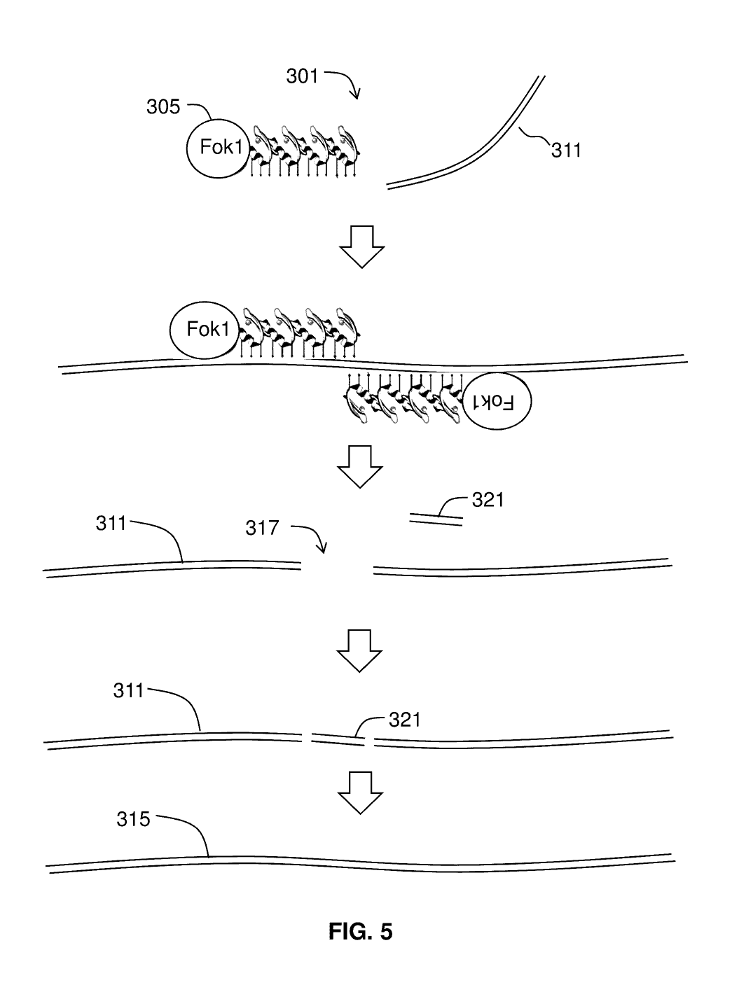

FIG. 5 shows ZFN being used to cut viral nucleic acid. Briefly, the ZFN method includes introducing into the infected host cell at least one vector (e.g., RNA molecule) encoding a targeted ZFN 305 and, optionally, at least one accessory polynucleotide. See, e.g., U.S. Pub. 2011/0023144 to Weinstein, incorporated by reference The cell includes target sequence 311. The cell is incubated to allow expression of the ZFN 305, wherein a double-stranded break 317 is introduced into the targeted chromosomal sequence 311 by the ZFN 305. In some embodiments, a donor polynucleotide or exchange polynucleotide 321 is introduced. Swapping a portion of the viral nucleic acid with irrelevant sequence can fully interfere transcription or replication of the viral nucleic acid. Target DNA 311 along with exchange polynucleotide 321 may be repaired by an error-prone non-homologous end-joining DNA repair process or a homology-directed DNA repair process.

Typically, a ZFN comprises a DNA binding domain (i.e., zinc finger) and a cleavage domain (i.e., nuclease) and this gene may be introduced as mRNA (e.g., 5′ capped, polyadenylated, or both). Zinc finger binding domains may be engineered to recognize and bind to any nucleic acid sequence of choice. See, e.g., Qu et al., 2013, Zinc-finger-nucleases mediate specific and efficient excision of HIV-1 proviral DAN from infected and latently infected human T cells, Nucl Ac Res 41(16):7771-7782, incorporated by reference. An engineered zinc finger binding domain may have a novel binding specificity compared to a naturally-occurring zinc finger protein. Engineering methods include, but are not limited to, rational design and various types of selection. A zinc finger binding domain may be designed to recognize a target DNA sequence via zinc finger recognition regions (i.e., zinc fingers). See for example, U.S. Pat. Nos. 6,607,882; 6,534,261 and 6,453,242, incorporated by reference. Exemplary methods of selecting a zinc finger recognition region may include phage display and two-hybrid systems, and are disclosed in U.S. Pat. No. 5,789,538; U.S. Pat. No. 5,925,523; U.S. Pat. No. 6,007,988; U.S. Pat. No. 6,013,453; U.S. Pat. No. 6,410,248; U.S. Pat. No. 6,140,466; U.S. Pat. No. 6,200,759; and U.S. Pat. No. 6,242,568, each of which is incorporated by reference.

A ZFN also includes a cleavage domain. The cleavage domain portion of the ZFNs may be obtained from any suitable endonuclease or exonuclease such as restriction endonucleases and homing endonucleases. See, for example, Belfort & Roberts, 1997, Homing endonucleases: keeping the house in order, Nucleic Acids Res 25(17):3379-3388. A cleavage domain may be derived from an enzyme that requires dimerization for cleavage activity. Two ZFNs may be required for cleavage, as each nuclease comprises a monomer of the active enzyme dimer. Alternatively, a single ZFN may comprise both monomers to create an active enzyme dimer. Restriction endonucleases present may be capable of sequence-specific binding and cleavage of DNA at or near the site of binding. Certain restriction enzymes (e.g., Type IIS) cleave DNA at sites removed from the recognition site and have separable binding and cleavage domains. For example, the Type IIS enzyme FokI, active as a dimer, catalyzes double-stranded cleavage of DNA, at 9 nucleotides from its recognition site on one strand and 13 nucleotides from its recognition site on the other. The FokI enzyme used in a ZFN may be considered a cleavage monomer. Thus, for targeted double-stranded cleavage using a FokI cleavage domain, two ZFNs, each comprising a FokI cleavage monomer, may be used to reconstitute an active enzyme dimer. See Wah, et al., 1998, Structure of FokI has implications for DNA cleavage, PNAS 95:10564-10569; U.S. Pat. No. 5,356,802; U.S. Pat. No. 5,436,150; U.S. Pat. No. 5,487,994; U.S. Pub. 2005/0064474; U.S. Pub. 2006/0188987; and U.S. Pub. 2008/0131962, each incorporated by reference.

In the ZFN-mediated process, a double stranded break introduced into the target sequence by the ZFN is repaired, via homologous recombination with the exchange polynucleotide, such that the sequence in the exchange polynucleotide may be exchanged with a portion of the target sequence. The presence of the double stranded break facilitates homologous recombination and repair of the break. The exchange polynucleotide may be physically integrated or, alternatively, the exchange polynucleotide may be used as a template for repair of the break, resulting in the exchange of the sequence information in the exchange polynucleotide with the sequence information in that portion of the target sequence. Thus, a portion of the viral nucleic acid may be converted to the sequence of the exchange polynucleotide. ZFN methods can include using a vector to deliver a nucleic acid molecule encoding a ZFN and, optionally, at least one exchange polynucleotide or at least one donor polynucleotide to the infected cell.

Meganucleases are endodeoxyribonucleases characterized by a large recognition site (double-stranded DNA sequences of 12 to 40 base pairs); as a result this site generally occurs only once in any given genome. For example, the 18-base pair sequence recognized by the I-SceI meganuclease would on average require a genome twenty times the size of the human genome to be found once by chance (although sequences with a single mismatch occur about three times per human-sized genome). Meganucleases are therefore considered to be the most specific naturally occurring restriction enzymes. Meganucleases can be divided into five families based on sequence and structure motifs: LAGLIDADG, GIY-YIG, HNH, His-Cys box and PD-(D/E)XK. The most well studied family is that of the LAGLIDADG proteins, which have been found in all kingdoms of life, generally encoded within introns or inteins although freestanding members also exist. The sequence motif, LAGLIDADG, represents an essential element for enzymatic activity. Some proteins contained only one such motif, while others contained two; in both cases the motifs were followed by ˜75-200 amino acid residues having little to no sequence similarity with other family members. Crystal structures illustrates mode of sequence specificity and cleavage mechanism for the LAGLIDADG family: (i) specificity contacts arise from the burial of extended β-strands into the major groove of the DNA, with the DNA binding saddle having a pitch and contour mimicking the helical twist of the DNA; (ii) full hydrogen bonding potential between the protein and DNA is never fully realized; (iii) cleavage to generate the characteristic 4-nt 3′-OH overhangs occurs across the minor groove, wherein the scissile phosphate bonds are brought closer to the protein catalytic core by a distortion of the DNA in the central “4-base” region; (iv) cleavage occurs via a proposed two-metal mechanism, sometimes involving a unique “metal sharing” paradigm; (v) and finally, additional affinity and/or specificity contacts can arise from “adapted” scaffolds, in regions outside the core α/β fold. See Silva et al., 2011, Meganucleases and other tools for targeted genome engineering, Curr Gene Ther 11(1):11-27, incorporated by reference.

Some embodiments of the invention may utilize modified version of a nuclease. Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. Similar to the inactive dCas9 (RuvC- and HNH-), a Cas9 nickase is still able to bind DNA based on gRNA specificity, though nickases will only cut one of the DNA strands. The majority of CRISPR plasmids are derived from S. pyogenes and the RuvC domain can be inactivated by a D10A mutation and the HNH domain can be inactivated by an H840A mutation.

A single-strand break, or nick, is normally quickly repaired through the HDR pathway, using the intact complementary DNA strand as the template. However, two proximal, opposite strand nicks introduced by a Cas9 nickase are treated as a double strand break, in what is often referred to as a ‘double nick’ or ‘dual nickase’ CRISPR system. A double-nick induced double strain break can be repaired by either NHEJ or HDR depending on the desired effect on the gene target. At these double strain breaks, insertions and deletions are caused by the CRISPR/Cas9 complex. In an aspect of the invention, a deletion is caused by positioning two double strand breaks proximate to one another, thereby causing a fragment of the genome to be deleted.

In some embodiments, a nuclease is a directed RNA nuclease that cleaves RNA from viruses or viral transcripts. One targetable RNA nuclease system is the Type III-A CRISPR-Cas Csm complex of Thermus thermophilus (TtCsm). TtCsm is composed of five different protein subunits (Csm1-Csm5) with an uneven stoichiometry and a single crRNA of variable size (35-53 nt). The TtCsm crRNA content is similar to the Type III-B Cmr complex, indicating that crRNAs are shared among different subtypes. TtCsm cleaves complementary target RNAs at multiple sites. Unlike Type I complexes, interference by TtCsm does not proceed via initial base pairing by a seed sequence. For discussion see Staals et al., 2014, RNA Targeting by the type III-A CRISPR-Cas Csm complex of Thermus thermophiles, Molecular Cell 56(4):518-530, incorporated by reference. Thus aspects of the invention provide a non-human transgenic organism comprising a transgene, wherein the transgene comprises nucleic acid that encodes a targeting nuclease that can be activated to digest foreign RNA. The nuclease may be TtCsm or any other suitable targetable nuclease that cuts RNA.

In some embodiments, the invention includes the use of the Dicer, the RNA-induced silencing complex (RISC), or both. Dicer, also known as endoribonuclease Dicer or helicase with RNase motif, is an enzyme of the RNase III family. Dicer cleaves double-stranded RNA (dsRNA) and pre-microRNA (pre-miRNA) into short double-stranded RNA fragments called small interfering RNA and microRNA respectively. These fragments are approximately 20-25 base pairs long with a two-base overhang on the 3′ end. Dicer facilitates the activation of the RNA-induced silencing complex (RISC), which is essential for RNA interference. RISC has a catalytic component argonaute, which is an endonuclease capable of degrading messenger RNA (mRNA).

RISC is a multi-protein complex, specifically a ribonucleoprotein, which incorporates one strand of a double-stranded RNA (dsRNA) fragment, such as small interfering RNA (siRNA) or microRNA (miRNA). The single strand acts as a template for RISC to recognize complementary messenger RNA (mRNA) transcript. Once found, argonaute activates and cleaves the mRNA. This process is called RNA interference (RNAi) and provides for gene silencing and defense against viral infections.

The RNase III Dicer aids RISC in RNA interference by cleaving dsRNA into 21-23 nucleotide long fragments with a two-nucleotide 3′ overhang. These dsRNA fragments are loaded into RISC and each strand has a different fate based on the asymmetry rule phenomenon.

The strand with the less stable 5′ end is selected by the argonaute and integrated into RISC. This strand is known as the guide strand. The other strand, known as the passenger strand, is degraded by RISC. RISC uses the bound guide strand to target complementary 3′-untranslated regions (3′UTR) of mRNA transcripts via Watson-Crick base pairing. RISC can now regulate gene expression of the mRNA transcript in a number of ways. RISC degrades target mRNA which reduces the levels of transcript available to be translated by ribosomes. There are two main requirements for mRNA degradation to take place: a near-perfect complementary match between the guide strand and target mRNA sequence; and a catalytically active argonaute protein, called a ‘slicer’, to cleave the target mRNA. Also, RISC can modulate the loading of ribosome and accessory factors in translation to repress expression of the bound mRNA transcript. Translational repression only requires a partial sequence match between the guide strand and target mRNA. Translation can be regulated at the initiation step by preventing the binding of the eukaryotic translation initiation factor (eIF) to the 5′ cap. It has been noted RISC can adeadenylate the 3′ poly(A) tail which might contribute to repression via the 5′ cap. RISC may also prevent the binding of the 60S ribosomal subunit to the mRNA. Thus some aspects of the invention provide a non-human transgenic organism comprising a transgene, wherein the transgene comprises nucleic acid that encodes a targeting nuclease that can be activated to digest foreign RNA.

Embodiments of the invention use a components of the Dicer/RISC system that naturally occur in plants or provides for the expression of an orthologous complex. In some embodiments, a transgenic agricultural crop plant or livestock animal is provided with a transgene for one or more component of the Dicer/RISC system. During infection, an RNA-induced silencing complex (RISC) is programmed with viral short-interfering RNAs (siRNAs) to target the cognate viral RNA for degradation. A RISC complex gene may be taken from Nicotiana benthamiana and cloned into the transgenic organism. Discussion may be found in Ciomperlik et al., 2012, An antiviral RISC isolated from Tobaccco rattle virus-infected plants, Virology 412(1):117-124, incorporated by reference.

Argonaute proteins are a family of proteins that play a role in RNA silencing as a component of the RNA-induced silencing complex (RISC). The Argonaute of the archaeon Pyrococcus furiosus (PfAgo) uses small 5′-phosphorylated DNA guides to cleave both single stranded and double stranded DNA targets, and does not utilize RNA as guide or target.

NgAgo uses 5′ phosphorylated DNA guides (so called “gDNAs”) and appear to exhibit little preference for any certain guide sequences and thus may offer a general-purpose DNA-guided programmable nuclease. NgAgo does not require a PAM sequence, which contributes to flexibility in choosing a genomic target. NgAgo also appears to outperform Cas9 in GC-rich regions. NgAgo is only 887 amino acids in length. NgAgo randomly removes 1-20 nucleotides from the cleavage site specified by the gDNA. Thus, PfAgo and NgAgo represent potential DNA-guided programmable nucleases that may be modified for use as a composition of the invention.

### Targeting Sequence

The transgenic organism may express a targeting nuclease that uses a targeting sequence such as a guide RNA (gRNA) to target and digest foreign nucleic acid while avoiding off-target (e.g., self) digestion. The invention provides methods to avoid self genome digestion. A targeting sequence may be pre-determined (e.g., to protect against a specific agricultural virus) and encoded within the transgene.

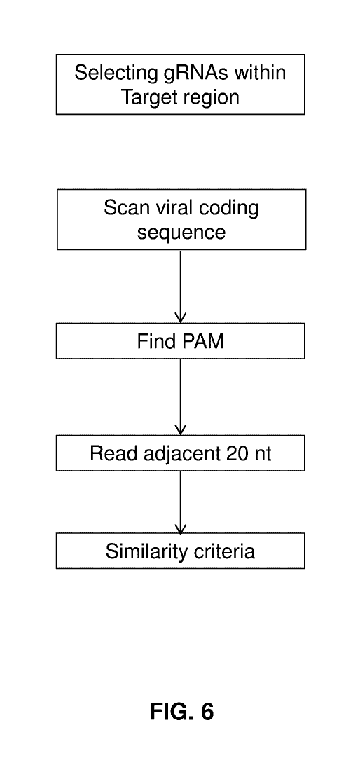

FIG. 6 describes an exemplary method for selecting a gRNA within the viral target region. A system or method of the invention may be used to scan the viral coding sequence and finds the PAM for the nuclease that is to be used. For example, where the digestion system will include cas9, the system scan the target for NGG, where N is any nucleotide. Upon finding the PAM in the viral genome, the 20 nucleotide string adjacent to the PAM within the viral genome are read. This 20 nucleotide string is provisionally treated as a potential sequence for the gRNA. Finally selecting the nucleotide string for the gRNA involves determining if the nucleotide string satisfies a similarity criteria for any region within the host genome (i.e., a gRNA is only selected if there is no region within the host genome that is similar according to a defined criteria).

Any suitable similarity criteria may be used. For example, one similarity criteria may be the requirement of a perfect match for all 20 bases of the nucleotide string. Other criteria may include that 19 bases match, or 18, etc. In a preferred embodiment, the invention includes similarity criteria that balance the requirement of actually finding a useful gRNA with the probabilities of some matching portions in the host, i.e., the possibility that even without a perfect 20 nt match, some of the gRNA may still bind to the host genome and initiate nuclease action. The includes similarity criteria that minimize off-target action against the host genome.

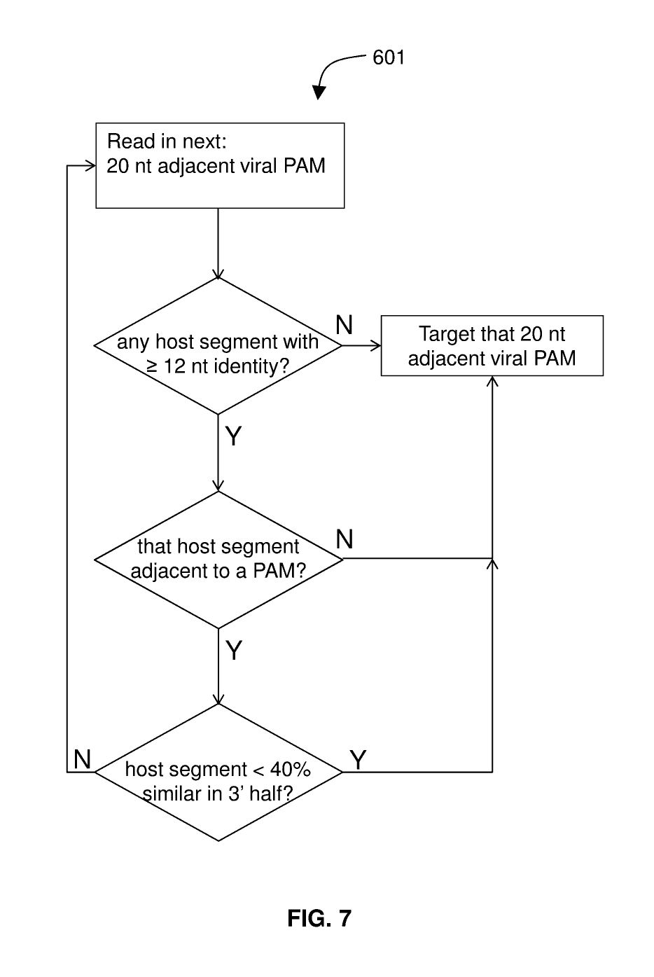

FIG. 7 outlines a similarity criteria 601 according to certain embodiments that may be automatically applied by, for example, a computer system. To avoid digestion of host genome, the system applies a search criteria that embodies certain principles. The system preferably tries to avoid any target sequence with any ≧12 nt DNA stretch homology to the human genome. When homology to human genome is inevitable, the guide RNA candidate not followed by PAM in the human genome would not lead to off-target digestion, and should be given priority. If homologous sequences and PAM both are present in the human genome, one should choose the guide RNA candidate with low homology (e.g., <40% similar) to human genome in the half next to PAM, where double strand break happens.

To reach these principles, as diagrammed in FIG. 7, the system reads in a 20 nt nucleotide string adjacent a PAM in the viral sequence. The system examines the host genome for any segment with ≧12 nt identity to the nucleotide string. If no such segment is found (N), then that nucleotide string is provided as the guide sequence to target that 20 nt in the viral genome. If such a segment is found in the human genome (Y), then the system determines if that segment in the host genome is adjacent to a PAM. If that segment in the host genome is not adjacent to a PAM (N), then that nucleotide string is provided as the guide sequence to target that 20 nt in the viral genome. If that segment in the host genome is adjacent to a PAM (Y), then the system determines if the half of that segment that is closest to the PAM is less than 40% similar to the nucleotide string. If the half of that segment that is closest to the PAM is less than 40% similar to the nucleotide string (Y), then that nucleotide string is provided as the guide sequence to target that 20 nt in the viral genome. If the half of that segment that is closest to the PAM is not less than 40% similar to the nucleotide string, then the system reads in the next 20 nt nucleotide string in the viral genome sequence that is adjacent to a PAM and repeats the steps on that next candidate string. The cycle of steps is optionally repeated until at least one guide sequence is provided. Optionally, the steps may be repeated until several or all possible guide sequences are provided.

In some embodiments of the invention, targeting sequences are expressed within an organelle such as a chloroplast. Expression within an organelle may be beneficial in protecting a gene, a plasmid, plastid, a gene product, etc., from deleterious elements such as endogenous plant RNAi pathways. In certain embodiments, a gene is provided within a chloroplast or other organelle that encodes nucleic acid that is complementary to a target gene or locus a virus, parasite, or pest such as an insect. Preferably, the gene is integrated into a genome of an organelle, such as the chloroplast genome or a mitochondrial genome. The nucleic acid may be expressed and used as a guide RNA, e.g., for a Cas9 enzyme (which may also be present as a gene or protein in the organelle or another organelle). It is also possible that the nucleic acid is a dsRNA that triggers a lethal RNAi response in a pest. See e.g., Zhang, 2015, Full crop protection from an insect pest by expression of long double-stranded RNAs in plastids, Science 347(6225):991-994, incorporated by reference. For example, an organelle may have a gene (either integrated into its genome or present as an independent particle such as a plasmid with its own replication origin) that when transcribed into RNA is complementary to a portion of a nucleic acid of a virus, parasite, or pest. The transcribed RNA hybridizes to the portion that it is complementary to and may trigger the RNAi system to destroy the target.

### Expression

Methods of the invention may be used to create a transgenic organism for agriculture that expresses a nuclease that digests foreign nucleic acid thus protecting the organism from viral infection. Any suitable expression pattern may be provided including, for example, constitutive or conditional expression.

In some embodiments, the full nuclease is constitutively expressed in all cells at all times. This may be beneficial for providing a transgenic crop plant or livestock animal with a form of immune system against viral infection. A CRISPR/Cas9 sequence may be constitutively expressed and may respond to viral infection by integrating fragments of viral nucleic acid into the clustered repeats of the CRISPR. Those then may function as template for guide RNAs during future infections.

In certain embodiments, the transgene or nuclease is only expressed in certain cells such as the cells that the virus is capable of infecting. An important characteristic of some viruses is tropism. Tropism of a virus pertains to the types of cells, tissues, and animal and plant species in which it can replicate. A transgene can be under control of a tissue-specific promoter, for example. Those include promoters controlling gene expression in a tissue-dependent manner and according to the developmental stage of the plant. The transgenes driven by these type of promoters will only be expressed in tissues where the transgene product is desired, leaving the rest of the tissues in the plant unmodified by transgene expression. Tissue-specific promoters may be induced by endogenous or exogenous factors, so they can be classified as inducible promoters as well. Unlike constitutive expression of genes, tissue-specific expression is the result of several interacting levels of gene regulation. As such, it is then preferable to use promoters from homologous or closely related plant species to achieve efficient and reliable expression of transgenes in particular tissues. Tissue promoters include beta-amylase gene or barley hordein gene promoters (for seed gene expression), tomato pz7 and pz130 gene promoters (for ovary gene expression), tobacco RD2 gene promoter (for root gene expression), banana TRX promoter and melon actin promoter (for fruit gene expression) and others. Tissue specific promoters may include root promoters such as those available from Pioneer Hi-Bred. Root promoters enhance or suppress the expression of a linked gene in root cells. Fruit promoters, such as those available from Calgene, include fruit specific promoters that control the expression of genes in mature ovary tissue of a fruit and in the receptacle tissue of accessory fruits such as strawberry, apple and pear. Seed promoters (e.g., available from Calgene) include transcription cassettes having a seed-specific promoter and recombinant molecules containing a seed-maturation promoter.

Additionally or alternatively, nuclease expression may be dependent on an external event. For example, a transgene may be under control of an inducible promoter linked to a small molecule. Numerous inducible promoters are known in the art and include chemically-regulated promoters such as those derived from organisms such as yeast, E. coli, Drosophila or mammals. Inducible promoters include alcohol-regulated promoters (e.g., available from Syngenta). These provide a transcriptional system containing the alcohol dehydrogenase I (alcA) gene promoter and the transactivator protein AlcR. Different agricultural alcohol-based formulations are used to control the expression of a gene of interest linked to the alcA promoter. In some embodiments, an inducible promoter is a tetracycline-regulated promoter, such as promoters available from BASF AG. The tetracycline-responsive promoter systems can function either to activate or repress gene expression system in the presence of tetracycline. Some of the elements of the systems include a tetracycline repressor protein (TetR), a tetracycline operator sequence (tetO) and a tetracycline transactivator fusion protein (tTA), which is the fusion of TetR and a herpes simplex virus protein 16 (VP16) activation sequence. Inducible promoters may include steroid-regulated promoters. Steroid-regulated promoters suitable for use include those based on the rat glucocorticoid receptor (GR), promoters based on the human estrogen receptor (ER), promoters based on ecdysone receptors derived from different moth species, as well as promoters from the steroid/retinoid/thyroid receptor superfamily. In some embodiments, an inducible promoter is metal-regulated. Promoters derived from metallothionein (proteins that bind and sequester metal ions) genes from yeast, mouse and human may be used to provide a promoter that is regulated by metal. Additionally, inducible promoters may include pathogenesis-related (PR) proteins that are induced in plants in the presence of particular exogenous chemicals in addition to being induced by pathogen infection. Salicylic acid, ethylene and benzothiadiazole (BTH) are some of the inducers of PR proteins. Such promoters have been derived from Arabidopsis and maize PR genes.

Using an inducible promoter provides control over conditional expression. For example, if a transgenic plant or animal appears sick or is exposed to infection, it may be administered a small molecule (e.g., via its feed) to initiate expression of the nuclease. Or this could be done periodically as a prophylactic measure. Thus in some aspects, the invention provides non-human transgenic organism comprising a transgene, wherein the transgene comprises nucleic acid that encodes a targeting nuclease that can be activated to digest foreign nucleic acid, wherein the transgene is under the control of a promoter. The promoter may be an inducible promoter, for example, a chemically-regulated promoters such as a tetracycline-regulated promoter. Additionally or alternatively, the promoter may be a virus-dependent promoter, so that nuclease expression is only turned on in infected cells.

For two component nucleases (like CRISPR) one of the two components may be expressed constitutively while the other is expressed conditionally (e.g., under the control of an inducible promoter). For example, the system may constitutively express CAS9 and have the guide RNA conditionally expressed (or vice-versa).

### Cleave Foreign Nucleic Acid

As discussed above, methods of the invention may be used to create a transgenic organism for agriculture that expresses a nuclease that digests foreign nucleic acid thus protecting the organism from viral infection. The nuclease may use a targeting sequencing that can also be transgenically included in the organism. Using the general methods described above, one may use knowledge of specific viral genomes to design targeting sequences against those viruses to so protect an organism.

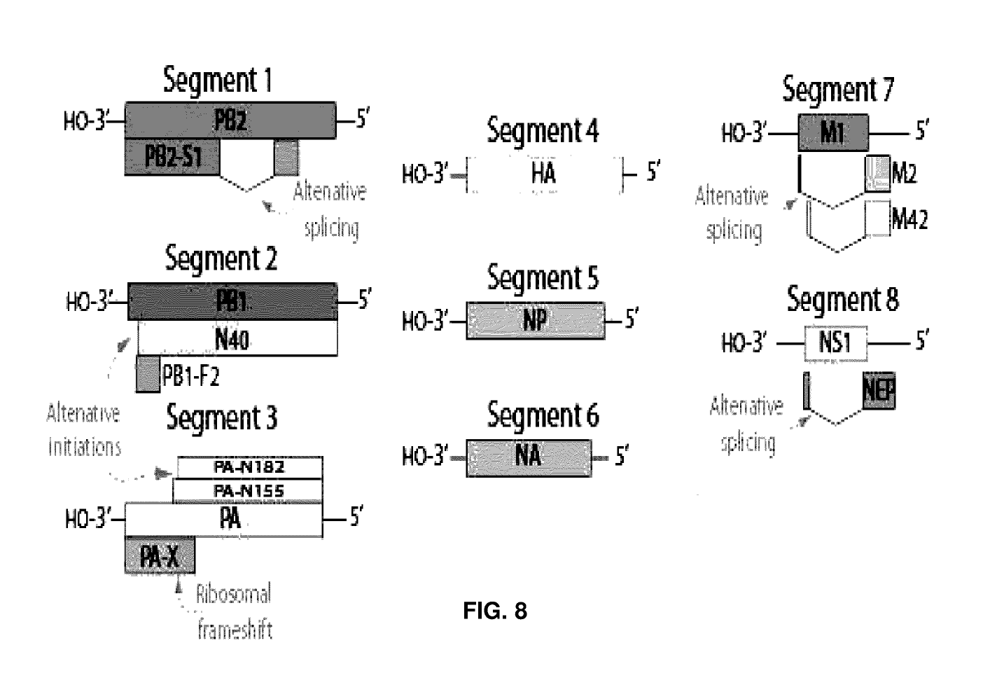

FIG. 8 diagrams the avian flu virus genome for targets for cleavage. The Avian flu virus genome has been described and published. See, e.g., Pabbaraju et al., 2014, Full-genome analysis of avian influenza A(H5N1) virus from a human, North America, 2013, Emerg Inf Dis 20(5):887-891, incorporated by reference. One my choose segments from that genome that meet the required similarity criteria against the native genome of the transgenic organism and include those segments as targeting sequences in a transgene. Thus, where the invention is used to provide a transgenic poultry organism, the animal can digest flu virus to avoid infection.

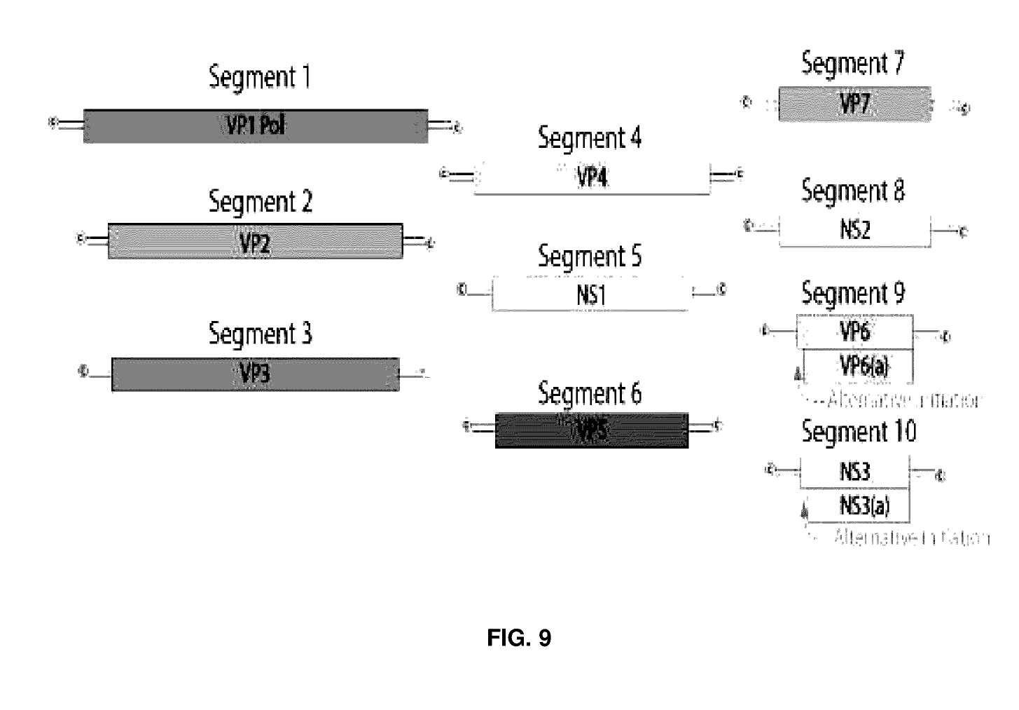

FIG. 9 shows a genome of a Bluetongue virus (BTV) for targeting. Bluetongue Virus (BTV) is a pathogenic virus that causes serious disease in livestock. The BTV genome has been described and published. See Minakshi et al., 2012, Complete genome sequence of Bluetongue virus serotype 16 of goat origin from India, J Virol 86(15):8337-8338, incorporated by reference. Using this information and the similarity criteria one may create a transgenic animal such as a sheep, cattle, or goat that itself digests Bluetongue virus genetic material.

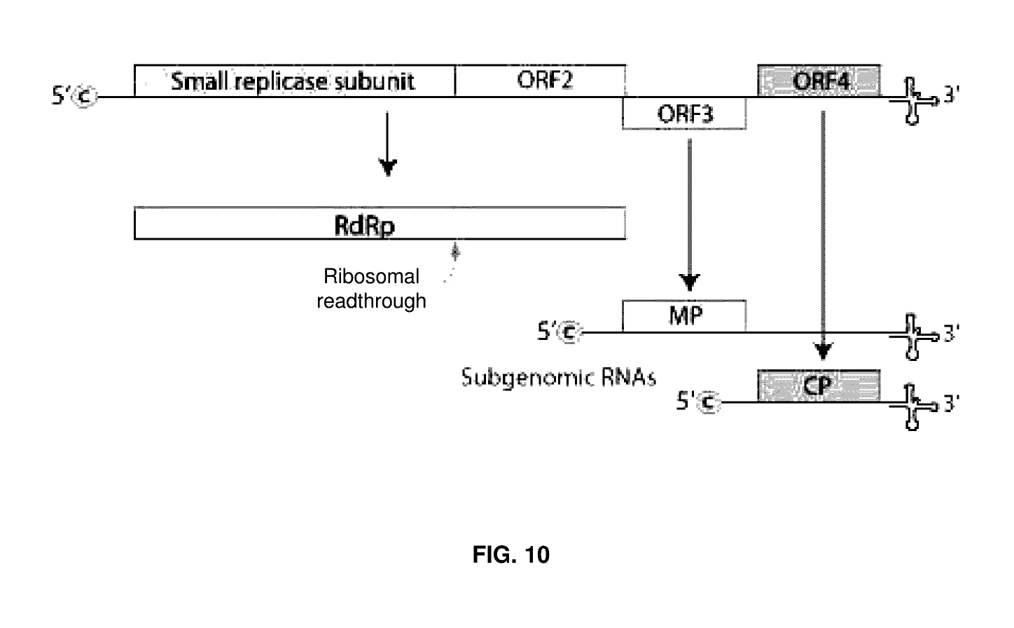

FIG. 10 diagrams the tobacco mosaic virus (TMV) genetic material. TMV infects a wide range of plants, especially tobacco and other members of the family Solanaceae. The TMV genome has been described. See Goelet et al., 1982, Nucleotide sequence of tobacco mosaic virus RNA, PNAS 79(19):5818-5822. A transgenic tobacco or other plant of the family Solanaceae may be created wherein the transgene encodes a targeting nuclease and a targeting sequence specific to the TMV genome, allowing the plant to digest the foreign nucleic acid.

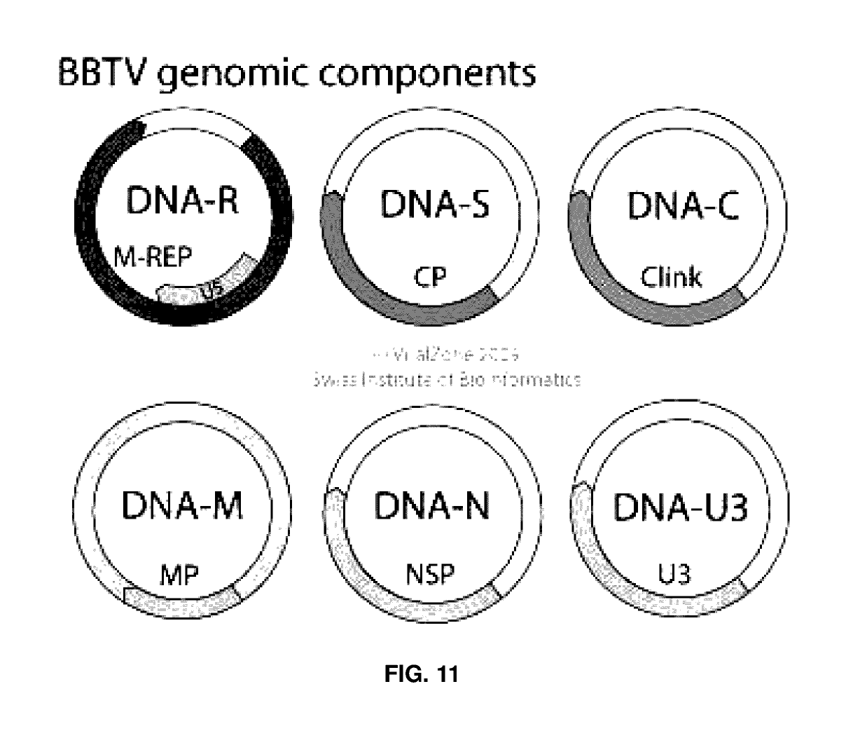

FIG. 11 shows parts of the genome of banana bunchy top virus (BBTV). BBTV is a major and significant disease that severely effects banana crops. The BBTV genome has been described. See Burns 1995, The genome organization of banana bunchy top virus: analysis of six ssDNA components, J Gen Vir 76:1471-1482, incorporated by reference. Using this information and the similarity criteria and methods described herein, methods of the invention may be used to provide a transgenic banana that digests BBTV genetic material.

Using methods, organisms, or seeds as provided by the invention, agriculture may be improved. For example, if livestock show signs of infection, they may be fed a small molecule that initiates transient expression of a nuclease to battle the infection. The nuclease may be expressed in every cell or in a tissue-specific manner. Additionally or alternatively, agricultural organisms could be treated prophylatically to prevent any signs of infection.

For additional background, see Yin et al., 2015, Multiplex conditional mutagenesis using transgenic expression of Cas9 and sgRNAs, Genetics 200:431-41; Xue et al., 2014, Efficient gene knock-out and knock-in with transgenic Cas9 in Drosophila, G3 4:925-929; and Harrison et al., 2014, A CRISPR view of development, Genes and Development 28:1859-1872, the contents of each of which are incorporated by reference.

In the CRISPR-Cas system, short sequences (referred to as “protospacers”) from an invading viral genome are copied as “spacers” between repetitive sequences in the CRISPR locus of the host genome. The CRISPR locus is transcribed and processed into short CRISPR RNAs (crRNAs) that guide the Cas to the complementary genomic target sequence. There are at least eleven different CRISPR-Cas systems, which have been grouped into three major types (I-III). In the type I and II systems, nucleotides adjacent to the protospacer in the targeted genome comprise the protospacer adjacent motif (PAM). The PAM is essential for Cas to cleave its target DNA, enabling the CRISPR-Cas system to differentiate between the invading viral genome and the CRISPR locus in the host genome, which does not incorporate the PAM. For additional details on this fascinating prokaryotic adaptive immune response, see recent reviews (Sorek et al. 2013; Terns and Terns 2014).

Transgenic expression of the gRNA has been demonstrated to increase the frequency of targeted events. The expression of Cas9 can be restricted by placing it under the control of tissue-specific regulatory sequences. Expression of Cas9 is discussed in Xue, 2014, Efficient gene knock-out and knock-in with transgenic Cas9 in Drosophila, G3 4(5):925-929 and in Yin, et al., 2015, Multiplex conditional mutagenesis using transgenic expression of Cas9 and sgRNAs, Genetics 200:431-441, both incorporated by reference. Agriculturally important viral targets may include RNA or ssDNA and Cas9 may be used to digest such nucleic acid. See O'Connell et al., 2014, Programmable RNA recognition and cleavage by CRISPR/Cas9, Nature 516:263-266; Price et al., Cas9-mediated targeting of viral RNA in eukaryotic cells, PNAS 112(19):6164-6169; Hwang et al., 2013, Heritable and precise zebrafish genome editing using a CRISPR-Cas system, PLoSOne 8(7):e68708, each incorporated by reference.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

### EXAMPLES

### Example 1

### Digesting Viral Nucleic Acid I

Methods and materials of the present invention may be used to digest foreign nucleic acid such as a genome of a hepatitis B virus (HBV).

It may be preferable to receive annotations for the HBV genome (i.e., that identify important features of the genome) and choose a candidate for targeting by enzymatic degredation that lies within one of those features, such as a viral replication origin, a terminal repeat, a replication factor binding site, a promoter, a coding sequence, and a repetitive region.

HBV, which is the prototype member of the family Hepadnaviridae, is a 42 nm partially double stranded DNA virus, composed of a 27 nm nucleocapsid core (HBcAg), surrounded by an outer lipoprotein coat (also called envelope) containing the surface antigen (HBsAg). The virus includes an enveloped virion containing 3 to 3.3 kb of relaxed circular, partially duplex DNA and virion-associated DNA-dependent polymerases that can repair the gap in the virion DNA template and has reverse transcriptase activities. HBV is a circular, partially double-stranded DNA virus of approximately 3200 by with four overlapping ORFs encoding the polymerase (P), core (C), surface (S) and X proteins. In infection, viral nucleocapsids enter the cell and reach the nucleus, where the viral genome is delivered. In the nucleus, second-strand DNA synthesis is completed and the gaps in both strands are repaired to yield a covalently closed circular DNA molecule that serves as a template for transcription of four viral RNAs that are 3.5, 2.4, 2.1, and 0.7 kb long. These transcripts are polyadenylated and transported to the cytoplasm, where they are translated into the viral nucleocapsid and precore antigen (C, pre-C), polymerase (P), envelope L (large), M (medium), S (small)), and transcriptional transactivating proteins (X). The envelope proteins insert themselves as integral membrane proteins into the lipid membrane of the endoplasmic reticulum (ER). The 3.5 kb species, spanning the entire genome and termed pregenomic RNA (pgRNA), is packaged together with HBV polymerase and a protein kinase into core particles where it serves as a template for reverse transcription of negative-strand DNA. The RNA to DNA conversion takes place inside the particles.

Numbering of basepairs on the HBV genome is based on the cleavage site for the restriction enzyme EcoR1 or at homologous sites, if the EcoR1 site is absent. However, other methods of numbering are also used, based on the start codon of the core protein or on the first base of the RNA pregenome. Every base pair in the HBV genome is involved in encoding at least one of the HBV protein. However, the genome also contains genetic elements which regulate levels of transcription, determine the site of polyadenylation, and even mark a specific transcript for encapsidation into the nucleocapsid. The four ORFs lead to the transcription and translation of seven different HBV proteins through use of varying in-frame start codons. For example, the small hepatitis B surface protein is generated when a ribosome begins translation at the ATG at position 155 of the adw genome. The middle hepatitis B surface protein is generated when a ribosome begins at an upstream ATG at position 3211, resulting in the addition of 55 amino acids onto the 5′ end of the protein.

ORF P occupies the majority of the genome and encodes for the hepatitis B polymerase protein. ORF S encodes the three surface proteins. ORF C encodes both the hepatitis e and core protein. ORF X encodes the hepatitis B X protein. The HBV genome contains many important promoter and signal regions necessary for viral replication to occur. The four ORFs transcription are controlled by four promoter elements (preS1, preS2, core and X), and two enhancer elements (Enh I and Enh II). All HBV transcripts share a common adenylation signal located in the region spanning 1916-1921 in the genome. Resulting transcripts range from 3.5 nucleotides to 0.9 nucleotides in length. Due to the location of the core/pregenomic promoter, the polyadenylation site is differentially utilized. The polyadenylation site is a hexanucleotide sequence (TATAAA) as opposed to the canonical eukaryotic polyadenylation signal sequence (AATAAA). The TATAAA is known to work inefficiently, suitable for differential use by HBV.

There are four known genes encoded by the genome, called C, X, P, and S. The core protein is coded for by gene C (HBcAg), and its start codon is preceded by an upstream in-frame AUG start codon from which the pre-core protein is produced. HBeAg is produced by proteolytic processing of the pre-core protein. The DNA polymerase is encoded by gene P. Gene S is the gene that codes for the surface antigen (HBsAg). The HBsAg gene is one long open reading frame but contains three in-frame start (ATG) codons that divide the gene into three sections, pre-S1, pre-S2, and S. Because of the multiple start codons, polypeptides of three different sizes called large, middle, and small (pre-S1+pre-S2+S, pre-S2+S, or S) are produced. The function of the protein coded for by gene X is not fully understood but it is associated with the development of liver cancer. It stimulates genes that promote cell growth and inactivates growth regulating molecules.

HBV starts its infection cycle by binding to the host cells with PreS1. Guide RNA against PreS1 locates at the 5′ end of the coding sequence. Endonuclease digestion will introduce insertion/deletion, which leads to frame shift of PreS1 translation. HBV replicates its genome through the form of long RNA, with identical repeats DR1 and DR2 at both ends, and RNA encapsidation signal epsilon at the 5′ end. The reverse transcriptase domain (RT) of the polymerase gene converts the RNA into DNA. Hbx protein is a key regulator of viral replication, as well as host cell functions. Digestion guided by RNA against RT will introduce insertion/deletion, which leads to frame shift of RT translation. Guide RNAs sgHbx and sgCore can not only lead to frame shift in the coding of Hbx and HBV core protein, but also deletion the whole region containing DR2-DR1-Epsilon. The four sgRNA in combination can also lead to systemic destruction of HBV genome into small pieces.

HBV replicates its genome by reverse transcription of an RNA intermediate. The RNA templates is first converted into single-stranded DNA species (minus-strand DNA), which is subsequently used as templates for plus-strand DNA synthesis. DNA synthesis in HBV use RNA primers for plus-strand DNA synthesis, which predominantly initiate at internal locations on the single-stranded DNA. The primer is generated via an RNase H cleavage that is a sequence independent measurement from the 5′ end of the RNA template. This 18 nt RNA primer is annealed to the 3′ end of the minus-strand DNA with the 3′ end of the primer located within the 12 nt direct repeat, DR1. The majority of plus-strand DNA synthesis initiates from the 12 nt direct repeat, DR2, located near the other end of the minus-strand DNA as a result of primer translocation. The site of plus-strand priming has consequences. In situ priming results in a duplex linear (DL) DNA genome, whereas priming from DR2 can lead to the synthesis of a relaxed circular (RC) DNA genome following completion of a second template switch termed circularization. It remains unclear why hepadnaviruses have this added complexity for priming plus-strand DNA synthesis, but the mechanism of primer translocation is a potential therapeutic target. As viral replication is necessary for maintenance of the hepadnavirus (including the human pathogen, hepatitis B virus) chronic carrier state, understanding replication and uncovering therapeutic targets is critical for limiting disease in carriers.

In some embodiments, systems and methods of the invention target the HBV genome by finding a nucleotide string within a feature such as PreS1.

Guide RNA against PreS1 locates at the 5′ end of the coding sequence. Thus it is a good candidate for targeting because it represents one of the 5′-most targets in the coding sequence. Endonuclease digestion will introduce insertion/deletion, which leads to frame shift of PreS1 translation. HBV replicates its genome through the form of long RNA, with identical repeats DR1 and DR2 at both ends, and RNA encapsidation signal epsilon at the 5′ end.

The reverse transcriptase domain (RT) of the polymerase gene converts the RNA into DNA. Hbx protein is a key regulator of viral replication, as well as host cell functions. Digestion guided by RNA against RT will introduce insertion/deletion, which leads to frame shift of RT translation.

Guide RNAs sgHbx and sgCore can not only lead to frame shift in the coding of Hbx and HBV core protein, but also deletion the whole region containing DR2-DR1-Epsilon. The four sgRNA in combination can also lead to systemic destruction of HBV genome into small pieces. In some embodiments, method of the invention include creating one or several guide RNAs against key features within a genome such as the HBV genome. To achieve the CRISPR activity in cells, expression plasmids coding cas9 and guide RNAs are delivered to cells of interest (e.g., cells carrying HBV DNA). To demonstrate in an in vitro assay, anti-HBV effect may be evaluated by monitoring cell proliferation, growth, and morphology as well as analyzing DNA integrity and HBV DNA load in the cells.

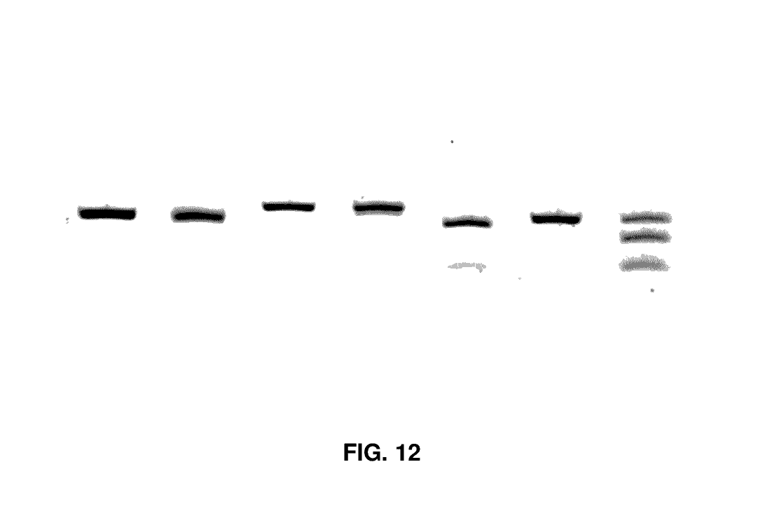

The described method may be validated using an in vitro assay. To demonstrate, an in vitro assay is performed with cas9 protein and DNA amplicons flanking the target regions. Here, the target is amplified and the amplicons are incubated with cas9 and a gRNA having the selected nucleotide sequence for targeting. As shown in FIG. 12, DNA electrophoresis shows strong digestion at the target sites.

FIG. 12 shows a gel resulting from an in vitro CRISPR assay against HBV. Lanes 1, 3, and 6: PCR amplicons of HBV genome flanking RT, Hbx-Core, and PreS1. Lane 2, 4, 5, and 7: PCR amplicons treated with sgHBV-RT, sgHBV-Hbx, sgHBV-Core, sgHBV-PreS1. The presence of multiple fragments especially visible in lanes 5 and 7 show that sgHBV-Core and sgHBV-PreS1 provide especially attractive targets in the context of HBV and that use of systems and methods of the invention may be shown to be effective by an in vitro validation assay.

### Example 2

### Digesting Viral Nucleic Acid II

An exemplary assay shows the digestion of viral nucleic acid.

Burkitt's lymphoma cell lines Raji, Namalwa, and DG-75 were obtained from ATCC and cultured in RPMI 1640 supplemented with 10% FBS and PSA, following ATCC recommendation. Human primary lung fibroblast IMR-90 was obtained from Coriell and cultured in Advanced DMEM/F-12 supplemented with 10% FBS and PSA.

Plasmids consisting of a U6 promoter driven chimeric guide RNA (sgRNA) and a ubiquitous promoter driven Cas9 were obtained from addgene, as described by Cong L et al. (2013) Multiplex Genome Engineering Using CRISPR/Cas Systems. Science 339:819-823.

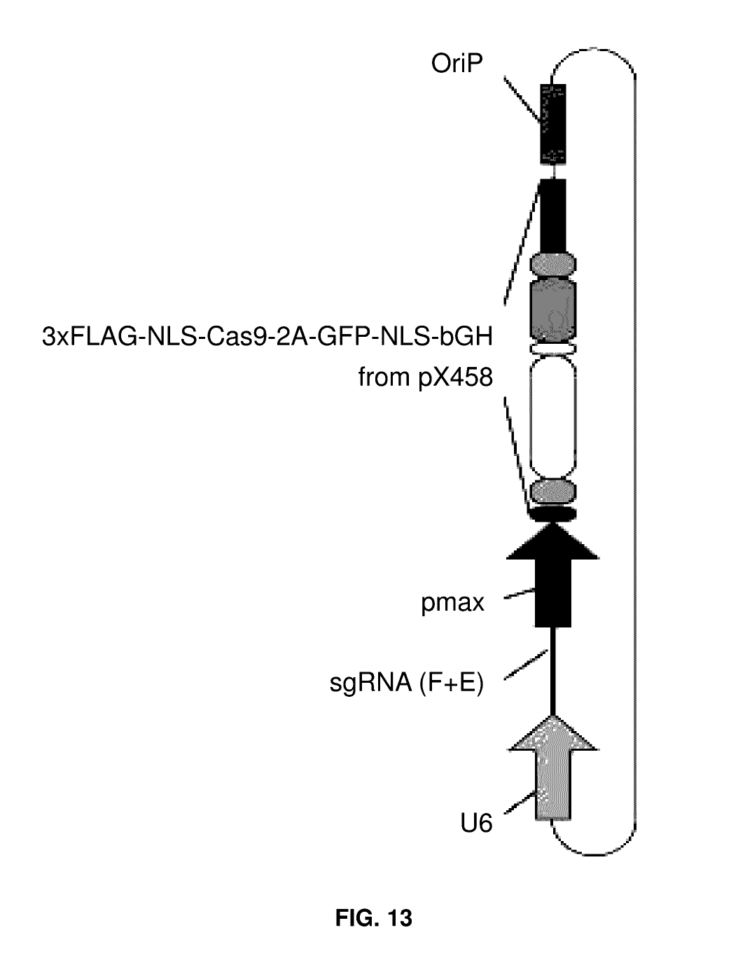

FIG. 13 shows a plasmid according to certain embodiments. An EGFP marker fused after the Cas9 protein allowed selection of Cas9-positive cells. A modified chimeric guide RNA stem-loop design was adapted for more efficient Pol-III transcription and more stable stem-loop structure (Chen B et al. (2013) Dynamic Imaging of Genomic Loci in Living Human Cells by an Optimized CRISPR/Cas System. Cell 155:1479-1491).

We obtained pX458 from Addgene, Inc. A modified CMV promoter with a synthetic intron (pmax) was PCR amplified from Lonza control plasmid pmax-GFP. A modified guide RNA sgRNA(F+E) was ordered from IDT. EBV replication origin oriP was PCR amplified from B95-8 transformed lymphoblastoid cell line GM12891. We used standard cloning protocols to clone pmax, sgRNA(F+E) and oriP to pX458, to replace the original CAG promoter, sgRNA and f1 origin. We designed EBV sgRNA based on the B95-8 reference, and ordered DNA oligos from IDT. The original sgRNA place holder in pX458 serves as the negative control.

Lymphocytes are known for being resistant to lipofection, and therefore we used nucleofection for DNA delivery into Raji cells. We chose the Lonza pmax promoter to drive Cas9 expression as it offered strong expression within Raji cells. We used the Lonza Nucleofector II for DNA delivery. 5 million Raji or DG-75 cells were transfected with 5 ug plasmids in each 100-ul reaction. Cell line Kit V and program M-013 were used following Lonza recommendation. For IMR-90, 1 million cells were transfected with 5 ug plasmids in 100 ul Solution V, with program T-030 or X-005. 24 hours after nucleofection, we observed obvious EGFP signals from a small proportion of cells through fluorescent microscopy. The EGFP-positive cell population decreased dramatically after that, however, and we measured <10% transfection efficiency 48 hours after nucleofection. We attributed this transfection efficiency decrease to the plasmid dilution with cell division. To actively maintain the plasmid level within the host cells, we redesigned the CRISPR plasmid to include the EBV origin of replication sequence, oriP. With active plasmid replication inside the cells, the transfection efficiency rose to >60%.

To design guide RNA targeting the EBV genome, we relied on the EBV reference genome from strain B95-8.

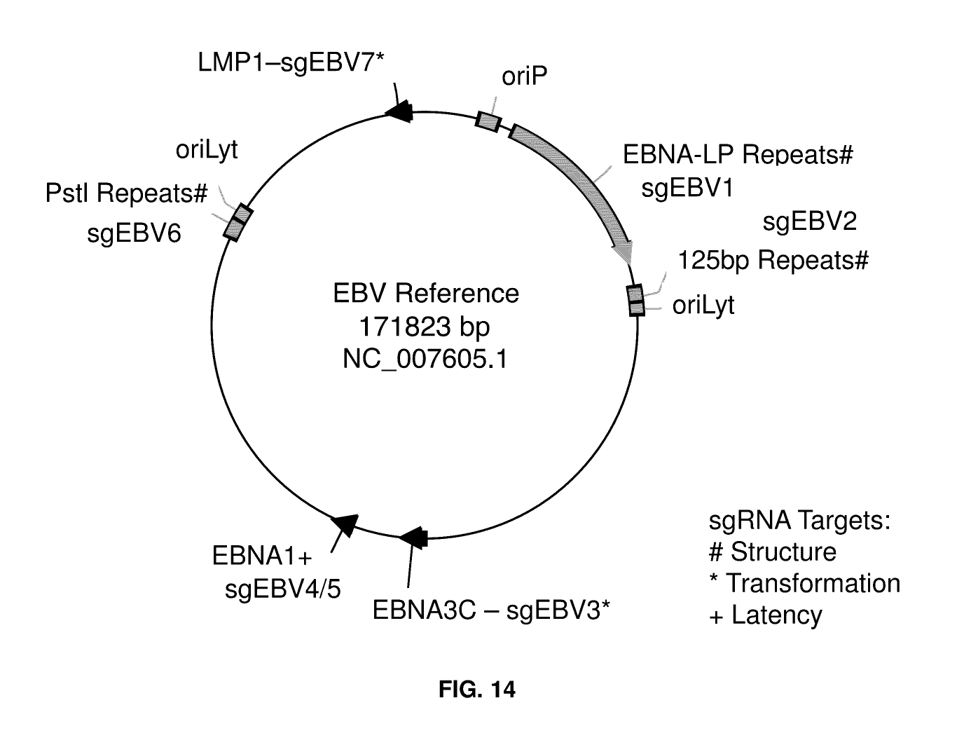

FIG. 14 diagrams the EBV genome. We targeted six regions with seven guide RNA designs for different genome editing purposes. The guide RNAs are listed in Table S1 in Wang and Quake, 2014, RNA-guided endonuclease provides a therapeutic strategy to cure latent herpesviridae infection, PNAS 111(36):13157-13162 and in the Supporting Information to that article published online at the PNAS website, and the contents of both of those documents are incorporated by reference for all purposes.

EBNA1 is crucial for many EBV functions including gene regulation and latent genome replication. We targeted guide RNA sgEBV4 and sgEBV5 to both ends of the EBNA1 coding region in order to excise this whole region of the genome. Guide RNAs sgEBV1, 2 and 6 fall in repeat regions, so that the success rate of at least one CRISPR cut is multiplied. These “structural” targets enable systematic digestion of the EBV genome into smaller pieces. EBNA3C and LMP1 are essential for host cell transformation, and we designed guide RNAs sgEBV3 and sgEBV7 to target the 5′ exons of these two proteins respectively.

### EBV Genome Editing

The double-strand DNA breaks generated by CRISPR are repaired with small deletions. These deletions will disrupt the protein coding and hence create knockout effects. SURVEYOR assays confirmed efficient editing of individual sites. Beyond the independent small deletions induced by each guide RNA, large deletions between targeting sites can systematically destroy the EBV genome.

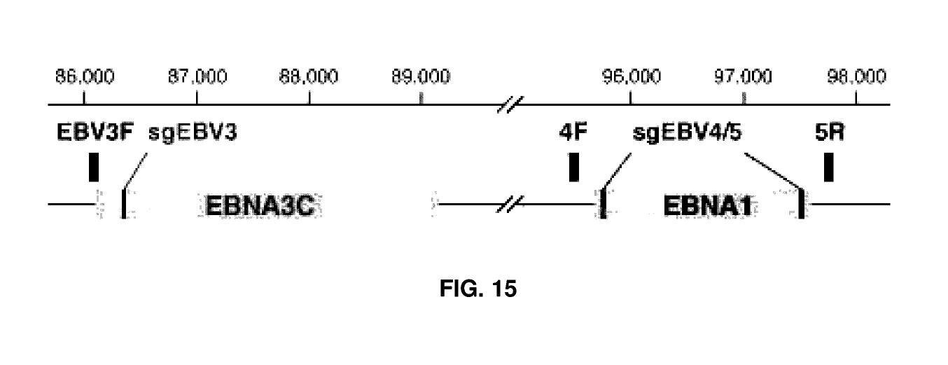

FIG. 15 shows genomic context around guide RNA sgEBV2 and PCR primer locations.

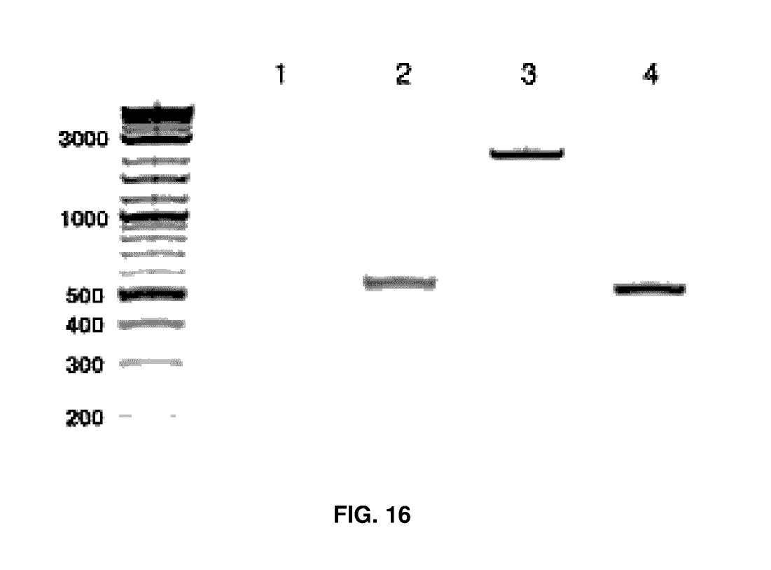

FIG. 16 shows a large deletion induced by sgEBV2, where lane 1-3 are before, 5 days after, and 7 days after sgEBV2 treatment, respectively. Guide RNA sgEBV2 targets a region with twelve 125-bp repeat units (FIG. 8). PCR amplicon of the whole repeat region gave a ˜1.8-kb band (FIG. 16). After 5 or 7 days of sgEBV2 transfection, we obtained ˜0.4-kb bands from the same PCR amplification (FIG. 16). The ˜1.4-kb deletion is the expected product of repair ligation between cuts in the first and the last repeat unit (FIG. 15).

DNA sequences flanking sgRNA targets were PCR amplified with Phusion DNA polymerase. SURVEYOR assays were performed following manufacturer's instruction. DNA amplicons with large deletions were TOPO cloned and single colonies were used for Sanger sequencing. EBV load was measured with Taqman digital PCR on Fluidigm BioMark. A Taqman assay targeting a conserved human locus was used for human DNA normalization. 1 ng of single-cell whole-genome amplification products from Fluidigm C1 were used for EBV quantitative PCR. We further demonstrated that it is possible to delete regions between unique targets (FIG. 10). Six days after sgEBV4-5 transfection, PCR amplification of the whole flanking region (with primers EBV4F and 5R) returned a shorter amplicon, together with a much fainter band of the expected 2 kb (FIG. 16).

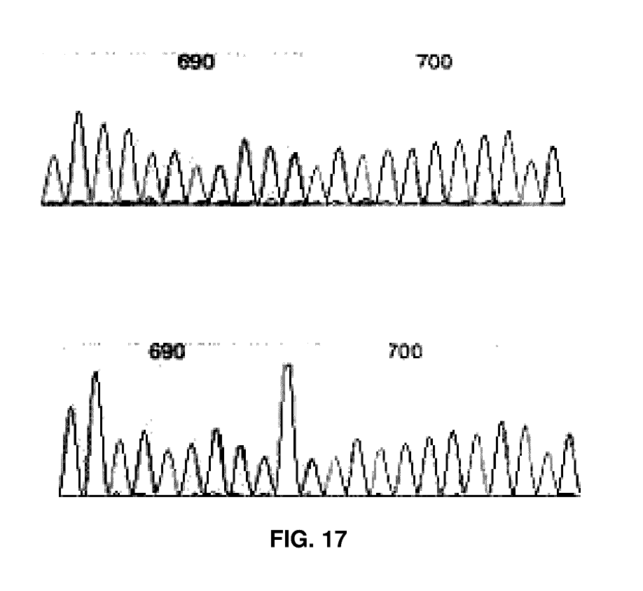

FIG. 17 shows that Sanger sequencing of amplicon clones confirmed the direct connection of the two expected cutting sites. A similar experiment with sgEBV3-5 also returned an even larger deletion, from EBNA3C to EBNA1. Additional information such as primer design is shown in Wang and Quake, 2014, RNA-guided endonuclease provides a therapeutic strategy to cure latent herpesviridae infection, PNAS 111(36):13157-13162 and in the Supporting Information to that article published online at the PNAS website, and the contents of both of those documents are incorporated by reference for all purposes.

Essential Targets For EBV Treatment. The seven guide RNAs in our CRISPR cocktail target three different categories of sequences which are important for EBV genome structure, host cell transformation, and infection latency, respectively. To understand the most essential targets for effective EBV treatment, we transfected Raji cells with subsets of guide RNAs. Although sgEBV4/5 reduced the EBV genome by 85%, they could not suppress cell proliferation as effectively as the full cocktail. Guide RNAs targeting the structural sequences (sgEBV1/2/6) could stop cell proliferation completely, despite not eliminating the full EBV load (26% decrease). We conclude that systematic destruction of EBV genome structure appears to be more effective than targeting specific key proteins for EBV treatment.

What is claimed is:

1. A non-human transgenic organism comprising a transgene, wherein the transgene comprises nucleic acid that encodes a targeting nuclease that can be activated to digest foreign nucleic acid.

2. The organism of claim 1, further comprising a feature that promotes expression of the transgene.

3. The organism of claim 2, wherein the feature that promotes expression comprises a promoter that selectively favors expression of the targeting nuclease within a certain tissue or cell type of the organism.

4. The composition of claim 2, wherein the nuclease is one selected from the group consisting of a zinc-finger nuclease, a transcription activator-like effector nuclease, and a meganuclease.

5. The organism of the claim 1, wherein the organism is a plant crop or mammalian livestock, and further wherein the targeting nuclease uses a targeting sequence to target and digest the foreign nucleic acid without digesting a genome of the organism.

6. The organism of claim 5, wherein the nuclease comprises Cas9 endonuclease and the targeting sequence comprises a guide RNA.

7. The organism of claim 6, wherein the guide RNA has no match according to predetermined similarity criteria within the genome.

8. The organism of claim 7, wherein the predetermined similarity criteria require that the guide RNA has no match >60% within the genome.

9. The organism of claim 5, wherein the targeting sequence is encoded adjacent the targeting nuclease within a complex in the transgene, and the complex is transcribed together as a single primary transcript.

10. The organism of claim 9, wherein activation of the targeting nuclease includes causing the complex to be transcribed.

11. The organism of claim 1, wherein activating the targeting nuclease includes administering an agent to cause expression of the transgene.

12. The organism of claim 1, wherein activating the targeting nuclease includes causing expression of the targeting nuclease from the transgene and causing the targeting nuclease to digest viral foreign nucleic acid.

13. The organism of claim 1, wherein the organism is a plant.

14. The organism of claim 1, wherein the organism is an animal.

15. A method of making a non-human transgenic organism, the method comprising:
introducing into a cell a transgene encoding a targeting nuclease;
integrating the transgene into heritable genetic material of the cell; and
growing the cell into an organism for agricultural use, wherein cells of the organism include the transgene.

16. The method of claim 15, wherein the organism is a mammal and the cell comprises an oocyte or a cell of an embryo and wherein growing the cell into the organism includes transfer of the oocyte or embryo into a recipient female.

17. The method of claim 16, wherein the targeting nuclease comprises Cas9 endonuclease under control of a promoter.

18. The method of claim 15, wherein the organism is a plant and the targeting nuclease comprises Cas9 endonuclease.

19. The method of claim 15, wherein the organism is a plant crop or mammalian livestock and the targeting nuclease comprises Cas9 endonuclease.

20. The method of claim 19, wherein the transgene also encodes at least one guide sequence that, when transcribed into a guide RNA, guides the Cas9 endonuclease to digest nucleic acid foreign to the organism.
